(19) United States
(12) Patent Application Publication
(10) Pub. No.: US 2024/0302341 A1
(43) Pub. Date: Sep. 12, 2024

(54) BROAD HYBRID FOREST REGRESSION (BHFR)-BASED SOFT SENSOR METHOD FOR DIOXIN (DXN) EMISSION IN MUNICIPAL SOLID WASTE INCINERATION (MSWI) PROCESS

(71) Applicant: BEIJING UNIVERSITY OF TECHNOLOGY, Beijing (CN)

(72) Inventors: Jian TANG, Beijing (CN); Heng XIA, Beijing (CN); Canlin CUI, Beijing (CN); Junfei QIAO, Beijing (CN)

(21) Appl. No.: 18/276,179

(22) PCT Filed: Oct. 27, 2022
(86) PCT No.: PCT/CN2022/127864

§ 371 Date: Aug. 7, 2023

(30) Foreign Application Priority Data
Jan. 19, 2022 (CN) 2022100599845

(51) Int. Cl. G01N 33/00; G06N 20/20

(52) U.S. Cl. CPC G01N 33/0075; G06N 20/20

(57) ABSTRACT

A broad hybrid forest regression (BHFR)-based soft sensor method for DXN emission in a municipal solid waste incineration (MSWI) process, including: based on a broad learning system (BLS) framework, constructing a BHFR soft sensor model for small sample high-dimensional data by replacing a neuron with a non-differential base learner, where the BHFR soft sensor model includes a feature mapping layer, a latent feature extraction layer, a feature incremental layer and an incremental learning layer, and the method includes: mapping a high-dimensional feature; extracting a latent feature from a feature space of a fully connected hybrid matrix, and reducing model complexity and computation consumption based on an information measurement criterion; enhancing a feature representation capacity by training the feature incremental layer based on an extracted latent feature; and constructing the incremental learning layer based on an incremental learning strategy, obtaining a weight matrix with a Moore-Penrose pseudo-inverse, and implementing high-precision modeling.

### CROSS REFERENCE TO RELATED APPLICATION

This patent application is a national stage application of International Patent Application No. PCT/CN2022/127864, filed on Oct. 27, 2022, which claims the benefit and priority of Chinese Patent Application No. 2022100599845, filed with the China National Intellectual Property Administration on Jan. 19, 2022, the disclosure of which is incorporated by reference herein in its entirety as part of the present application.

### TECHNICAL FIELD

The present disclosure relates to the technical field of soft sensors for dioxin (DXN) emission, in particular to a broad hybrid forest regression (BHFR)-based soft sensor method for DXN emission in a municipal solid waste incineration (MSWI) process.

### BACKGROUND

Municipal solid waste incineration (MSWI), a global solution to “waste besieging cities” at present, features safe disposal, waste reduction and resource recycling. MSWI will emit dioxin (DXN) and is required to minimize such emission since DXN is a persistent and highly toxic organic pollutant organized waste gas, and causes the phenomenon of “not-in-my-back-yard (NIMBY)” in the case of constructing incineration plants. An off-line assay and analysis method based on high resolution gas chromatography/high-resolution mass spectrometry (HRGC/HRMS) is a current central tool for measuring a DXN emission concentration. The method has the downside of great technical difficulty, long time lag and high labor and economic cost, and becomes one key factor that hinders real-time optimal control over the MSWI process in turn. To this end, on-line measurement of the DXN emission concentration has become a foremost challenge during MSWI.

Attention has been focused on an online indirect measurement method for a DXN concentration by constructing a correlation model with DXN related substances that can be measured online for solving the above problems. However, the method has some drawbacks, such as apparatus complexity, high costs, susceptibility to interference, and unpredictable prediction accuracy as a measurement method combining data modeling in essence. Compared with off-line analysis and on-line indirect measurement methods, soft sensor technology driven by easy-to-measure process data collected through an industrial distributed control system can effectively solves the problem that the DXN concentration cannot be measured online. With stability, accuracy and quick responses, the soft sensor technology has been extensively applied to difficult-to-measure parameters in complex industrial processes of petroleum, chemical industry, steelmaking, etc.

### SUMMARY

An objective of the present disclosure is to provide a broad hybrid forest regression (BHFR)-based soft sensor method for DXN emission in a municipal solid waste incineration (MSWI) process. A soft sensor modeling algorithm based on broad hybrid forest regression (BHFR) is provided with measurement of a DXN emission concentration in the MSWI process as the target.

To achieve the above objective, the present disclosure provides the following technical solutions.

A broad hybrid forest regression (BHFR)-based soft sensor method for dioxin (DXN) emission in a municipal solid waste incineration (MSWI) process includes: based on a broad learning system (BLS) framework, constructing a BHFR soft sensor model for small sample high-dimensional data by replacing a neuron with a non-differential base learner, where the BHFR soft sensor model includes a feature mapping layer, a latent feature extraction layer, a feature incremental layer and an incremental learning layer, and the method specifically includes:
S1, constructing the feature mapping layer, and mapping a high-dimensional feature by constructing a hybrid forest group composed of a random forest (RF) and a completely random forest (CRF);
S2, constructing the latent feature extraction layer, extracting a latent feature from a feature space of a fully connected hybrid matrix according to a contribution rate, guaranteeing maximum transmission and minimum redundancy of potential valuable information based on an information measurement criterion, and reducing model complexity and computation consumption;
S3, constructing the feature incremental layer, and further enhancing a feature representation capacity by training the feature incremental layer based on an extracted latent feature;
S4, constructing the incremental learning layer based on an incremental learning strategy, obtaining a weight matrix with a Moore-Penrose pseudo-inverse, and implementing high-precision modeling of the BHFR soft sensor model;
S5, verifying the soft sensor model by using n industrial process DXN data set; and
S6, using a soft sensor on DXN emission in the MSWI process with the soft sensor model constructed in steps S1-S5.

Further, S1 of constructing the feature mapping layer, and mapping a high-dimensional feature by constructing a hybrid forest group composed of a RF and a CRF specifically includes:
setting original data as {X,y}, where XϵR^{N}^{_{Raw}}^{×M} represents original input data, N_{Raw} represents a number of the original data, M represents a dimension of the original input data, is sourced from six different stages in the MSWI process, and is collected and stored in a distributed control system (DCS) in seconds, and yϵR^{N}^{_{Raw}}^{×1} represents an output truth of a DXN emission concentration, and is sourced from an emission DXN measurement sample obtained through an off-line measurement method; describing a modeling process of the feature mapping layer by taking a nth hybrid forest group of feature mapping layer as an example:
obtaining J training subsets of a hybrid forest group model by performing Bootstrap and random subspace (RSM) sampling on as follows:

$${\{ {X}_{Bootstrap}^{n , j} , {y}_{Bootstrap}^{n , j} \}}_{j = 1}^{J} = {φ}_{n}^{FML} ( {ϕ}_{n}^{FML} ( ( X , y ) , {P}_{Bootstrap} ) ) \qquad ( 1 )$$

where X_{Bootstrap}^{n,j} and y_{Bootstrap}^{n,j} represent an input and an output of a J training subset respectively, ϕₙ^{FML}(⋅) and φₙ^{FML}(⋅) represent Bootstrap sampling and RSM sampling of the nth hybrid forest group in the feature mapping layer respectively, and P_{Bootstrap} represents a Bootstrap sampling probability;
training a hybrid forest algorithm including J decision trees based on {X_{Bootstrap}^{n,j},y_{Bootstrap}^{n,j}}ⱼ₌₁^{J}, where a jth decision tree of the nth hybrid forest group in the feature mapping layer is expressed as follows:

$${f}_{n , j}^{DT} ( \cdot ) = {{\sum }^{L}}_{l = 1} {c}_{l} I ( {x}_{Bootstrap}^{n , j} ∈ {R}_{l} ) , l = 1 , 2 , … , L \qquad ( 2 )$$

where L represents a number of leaf nodes of the decision tree, I(⋅) represents an indicator function, and cₗ is computed through recursive splitting;
expressing a splitting loss function ⋅ᵢ(⋅) of the decision tree in the RF as:

$${Ω}_{i} ( s , v ) = min ( [ {y}_{L} - E [ {y}_{L} ] ] + [ {y}_{R} - E [ {y}_{R} ] ] ) = min ( {\sum }_{{x}_{Bootstrap}^{n , j} ∈ {R}_{L}} {( {y}_{L}^{i} - {c}_{L} )}^{2} + {\sum }_{{x}_{Bootstrap}^{n , j} ∈ {R}_{R}} {( {y}_{R}^{i} - {c}_{R} )}^{2} ) \qquad ( 3 )$$

where Ωᵢ(s,v) represents a value V of a sth feature value taken as a loss function value of a splitting criterion, y_{L} represents a truth vector of a DXN emission concentration at a left leaf node, E[y_{L}] represents a mathematical expectation of y_{L}, y_{R} represents a truth vector of a DXN emission concentration at a right leaf node, E[y_{R}] represents a mathematical expectation of y_{R}, y_{L}ⁱ represents a ith DXN emission concentration truth at the left leaf node, y_{R}ⁱ represents a ith DXN emission concentration truth at the right leaf node, c_{L} represents a predicted output of the DXN emission concentration at the left leaf node, and c_{R} represents a predicted output of the DXN emission concentration at the right leaf node;

splitting, by minimizing Ωᵢ(s,v), a training set (X_{Bootstrap}^{n,j},y_{Bootstrap}^{n,j}) into two tree nodes as follows:

$$min {\{ {Ω}_{i} ( s , v ) \}}_{i = 1}^{{N}_{Raw} \times M} {⟶}^{Tree node splitting} \{ \begin{matrix}{R}_{L}^{{N}_{L} \times M} \\ {R}_{R}^{{N}_{R} \times M}\end{matrix} \qquad ( 4 )$$

where R_{L}^{N}^{_{L}}^{×M} and R_{R}^{N}^{_{R}}^{×M} represent sample sets included in a left tree node and a right tree node after division respectively, N_{L} and N_{R} represents a number of samples in R_{L}^{N}^{_{L}}^{×M} and R_{R}^{N}^{_{R}}^{×M} respectively;
expressing predicted output values c_{L}^{RF} and c_{R} of the DXN emission concentration at a current left tree node and a current right tree node as sample truth expectations as follows:

$$\{ \begin{matrix}{c}_{L}^{RF} = E [ {y}_{L} ] , & {y}_{L} ∈ {R}_{L}^{{N}_{L} \times M} \\ {c}_{R}^{RF} = E [ {y}_{R} ] , & {y}_{R} ∈ {R}_{R}^{{N}_{R} \times M}\end{matrix} \qquad ( 5 )$$

where y_{L} and y_{R} represent truth vectors of the DXN emission concentration in R_{L}^{N}^{_{L}}^{×M} and R_{R}^{N}^{_{R}}^{×M}, and E[y_{L}] and E[y_{R}] represent mathematical expectations of y_{L} and y_{R};
different from the RF, expressing a splitting loss function in the CRF in a completely random selection mode as follows:

$$rand {\{ {( s , v )}_{i} \}}_{i = 1}^{{N}_{Raw} \times M} {⟶}^{Tree node splitting} \{ \begin{matrix}{R}_{L}^{{N}_{L} \times M} \\ {R}_{R}^{{N}_{R} \times M}\end{matrix} \qquad ( 6 )$$

where rand{(s,v)ᵢ}ᵢ₌₁^{N}^{_{Raw}}^{×M} represents that a value v of a sth feature is completely randomly selected as a split point;
expressing predicted output values c_{L}^{CRF} and c_{R}^{CRF} of the DXN emission concentration at a left tree node and a right tree node that are randomly split as sample truth expectations as follows:

$$\begin{matrix}{c}_{L}^{CRF} = E [ {y}_{L} ] , & {y}_{L} ∈ {R}_{L}^{{N}_{L} \times M} \\ {c}_{R}^{CRF} = E [ {y}_{R} ] , & {y}_{R} ∈ {R}_{R}^{{N}_{R} \times M}\end{matrix} \qquad ( 7 )$$

expressing, through the above process, the nth hybrid forest group fₙ^{FML}(⋅) as follows:

$${f}_{n}^{FML} ( \cdot ) = \{ {f}_{n , RF}^{FML} ( \cdot ) , {f}_{n , CRF}^{FML} ( \cdot ) \} \qquad ( 8 )$$

where f_{n,RF}^{FML}(⋅) represents a nth random forest, and f_{n,CRF}^{FML}(⋅) represents a nth completely random forest;
expressing a nth mapped feature Zₙ as follows:

$${Z}_{n} = {f}_{n}^{FML} ( X ) = \{ {f}_{n , RF}^{FML} ( X ) , {f}_{n , CRF}^{FML} ( X ) \} = [ ( {c}_{1 , l}^{n , RF} , {c}_{1 , l}^{n , RF} ) , … , ( {c}_{{n}_{Raw} , l}^{n , RF} , {c}_{{n}_{Raw} , l}^{n , RF} ) , … , ( {c}_{{N}_{Raw} , l}^{n , RF} , {c}_{{N}_{Raw} , l}^{n , RF} ) ] \qquad ( 9 )$$

where (c_{1,l}^{n,RF},c_{1,l}^{n,RF}) represents a mapped feature, obtained through the nth hybrid forest group, of a first sample of original input data from six different stages in the MSWI process, (cₙ_{_{Raw}}_{,l}^{n,RF},cₙ_{_{Raw}}_{,l}^{n,RF}) represents a mapped feature, obtained through the nth hybrid forest group, of a n_{Raw}th sample of original input data from six different stages in the MSWI process, and (c_{N}_{_{Raw}}_{,l}^{n,RF},c_{N}_{_{Raw}}_{,l}^{n,RF}) represents a mapped feature, obtained through the nth hybrid forest group, of a N_{Raw}th sample of original input data from six different stages in the MSWI process; and
expressing an output of the feature mapping layer as:

$${Z}^{N} = ( {Z}_{1} , {Z}_{2} , … , {Z}_{N} ) ∈ {R}^{{N}_{R a w} \times 2 N} \qquad ( 10 )$$

where Z₁₁ represents a first mapped feature, Z₂ represents a second mapped feature, Z_{N} represents a Nth mapped feature, and a mapped feature matrix Z^{N} includes N_{Raw} samples and a 2N dimensional feature.

Further, S2 of constructing the latent feature extraction layer, extracting a latent feature from a feature space of a fully connected hybrid matrix according to a contribution rate, guaranteeing maximum transmission and minimum redundancy of potential valuable information based on an information measurement criterion, and reducing model complexity and computation consumption specifically includes:
obtaining and expressing a fully connected hybrid matrix A by combining original input data X from six different stages in the MSWI process with a mapped feature matrix Z^{N} as follows:

$$A = [ X \vert {Z}^{N} ] ∈ {R}^{{N}_{{R}_{aw}} \times ( M + 2 N )} \qquad ( 11 )$$

where A includes N_{Raw} samples and a (M+2N) dimensional feature;
considering that a dimension of A is much higher than a dimension of the original data, minimizing redundant information in A through principal component analysis (PCA), and computing a correlation matrix R of A as follows:

$$R = \frac{1}{{N}_{Raw} - 1} {A}^{T} A ∈ {R}^{( M + 2 N ) \times ( M + 2 N )} \qquad ( 12 )$$

obtaining (M+2N) feature values and corresponding feature vectors by performing singular value decomposition on R as follows:

$$R = {U}_{( M + 2 N )} {\sum }_{( M + 2 N )} {V}_{( M + 2 N )} \qquad ( 13 )$$

where U_{(M+2N)} represents a (M+2N) order orthogonal matrix, Σ_{(M+2N)} represents a (M+2N) order diagonal matrix, and V_{(M+2N)} represents a (M+2N) order orthogonal matrix;

$${\sum }_{( M + 2 N )} = [ \begin{matrix}{σ}_{1} & & \\ & ⋱ & \\ & & {σ}_{( M + 2 N )}\end{matrix} ] \qquad ( 14 )$$

where σ₁>σ₂> . . . >σ_{(M+2)} represents eigenvalue values arranged in a descending order;
determining a number of final principal components according to a set latent feature contribution threshold n,

$$η = {\sum }_{q = 1}^{{Q}_{P C A}} {σ}_{q} / {\sum }_{q = 1}^{( M + 2 N )} {σ}_{q} \qquad ( 15 )$$

where a number of latent features is Q_{PCA}«(M+2N);
based on Q_{PCA} determined latent features above, obtaining a feature vector matrix V_{Q}_{_{PCA}} (that is, a projection matrix of A) corresponding to an eigenvalue value set {σ_{q}}_{q=1}^{Q}^{_{PCA}}, minimizing redundant information by performing feature projection on A, and recording an obtained latent feature as X^{PCA}, that is,

$${X}^{PCA} = A {V}_{{I}_{PCA}} ∈ {R}^{{N}_{R a w} \times {M}_{PCA}} \qquad ( 16 )$$

where V_{Q}_{_{PCA}}ϵR^{(M+2N)×Q}^{_{PCA}} represents a eigenvector of front Q_{PCA} latent features;
computing a mutual information value I″ between a selected latent feature X^{PCA} and a truth yϵR^{N}^{_{Raw}}^{×1} as follows:

$${I}^{M I} ( {X}^{PCA} , y ) = {\sum }_{q = 1}^{{Q}_{PCA}} p ( {x}_{q}^{PCA} , y ) {log}_{2} \frac{p ( {x}_{q}^{PCA} , y )}{p ( {x}_{q}^{PCA} ) , p ( y )} \qquad ( 17 )$$

where p(x_{q}^{PCA}, y) represents a joint probability distribution of a qth qth latent feature x_{q}^{PCA} and a DXN emission concentration truth y, p(x_{q}^{PCA}) represents a marginal probability PCA distribution of the qth latent feature x_{q}^{PCA}, and p(y) represents a marginal probability distribution of the DXN emission concentration truth y;
guaranteeing a correlation between the selected latent feature and the truth through an information maximization selection mechanism, and expressing the correlation as:

$${\{ {I}_{q}^{MI} \}}_{q = 1}^{{Q}_{PCA}} {{I}_{q}^{MI} \geq ζ}_{\to } {\{ {I}_{q}^{MI} \}}_{q = 1}^{{Q}_{PCA}^{MI}} ; \qquad ( 18 )$$

where {I_{q}^{MI}}_{q=1}^{Q} represents a mutual information value between Q^{PCA} latent features x_{q}^{PCA} and the truth y, ζ represents an information maximization threshold, and

$${\{ {I}_{q}^{MI} \}}_{q = 1}^{{Q}_{PCA}^{MI}}$$

represents Q_{PCA}^{MI} latent features having a greatest correlation with information of the DXN emission concentration truth y; and
obtaining a new data set {X′, y}ϵR^{N}^{_{Raw}}^{×(Q}^{_{PCA}}^{^{MI}}⁺¹⁾ including Q_{PCA}^{MI} latent features, and setting a dimension M_{PCA}^{MI}=Q_{PCA}^{MI} after extraction.

Further, S3 of constructing the feature incremental layer, and further enhancing a feature representation capacity by training the feature incremental layer based on an extracted latent feature specifically includes:
obtaining a J training subset of a hybrid forest algorithm by performing Bootstrap sampling and RSM sampling on the new data set {X′,y} as follows:

$${\{ {X}_{Bootstrap}^{′ k , j} , {y}_{Bootstrap}^{k , j} \}}_{j = 1}^{J} = {φ}_{k}^{FEL} ( {ϕ}_{k}^{FEL} ( \{ {X}^{′} , y \} , {P}_{Bootstrap} ) ) \qquad ( 19 )$$

where X′_{Bootstrap}^{k,j} and y_{Bootstrap}^{k,j} represent an input and an output of the jth training subset, X′ and y represent an input and an output of a new training set respectively, ϕₖ^{FEL}(⋅) represents Bootstrap sampling on a kth hybrid forest group, and φₖ^{FEL}(⋅) represents RSM sampling on the kth hybrid forest group;
taking construction of a jth RF in the kth hybrid forest group as an example as follows:

$$\{ {X}_{Bootstrap}^{′ k , j} , {y}_{Bootstrap}^{k , j} \} {{Ω}_{j} ( s , v )}_{\to } {f}_{k , j}^{DT - RF} ( \cdot ) = {\sum }_{l = 1}^{L} {c}_{l} I ( {x}_{Bootstrap}^{k , j} ∈ {R}_{l} ) , \qquad ( 20 )$$

$$l = 1 , 2 , … , L$$

where f_{k,j}^{DT-RF}(⋅) represents a jth decision tree of the RF in the kth hybrid forest group in the feature incremental layer; L represents a number of leaf nodes of the decision tree; and cₗ is computed through recursive splitting with specific process formulas of (3)-(5);
obtaining an RF model in the kth hybrid forest group in the feature incremental layer and expressing the RF model as follows:

$${f}_{k , RF}^{FEL} ( \cdot ) = {\{ {f}_{k , j}^{DT - RF} ( \cdot ) \}}_{j = 1}^{J} \qquad ( 21 )$$

similarly taking construction of a jth CRF in the kth hybrid forest group as an example as follows:

$$\{ {X}_{Bootstrap}^{′ k , j} , {y}_{Bootstrap}^{k , j} \} {⟶}^{{rand}_{j} ( s , v )} {f}_{k , j}^{DT - CRF} ( \cdot ) = {\sum }_{l = 1}^{L} {c}_{l} I ( {x}_{Bootstrap}^{k , j} ∈ {R}_{l} ) , \\ l = 1 , 2 , … , L \qquad ( 22 )$$

where f_{k,j}^{DT-CRF}(⋅) represents a jth decision tree of the CRF in the kth hybrid forest group in the feature incremental layer; and cₗ is computed through recursive splitting with specific process formulas of (6)-(7);
obtaining a CRF model in the kth hybrid forest group in the feature incremental layer and expressing the CRF model as follows:

$${f}_{k , CRF}^{FEL} ( \cdot ) = {\{ {f}_{k , j}^{DT - CRF} ( \cdot ) \}}_{j = 1}^{J} \qquad ( 23 )$$

obtaining the kth hybrid forest group fₖ^{FEL}(⋅) through the above process, and expressing a kth enhanced feature as follows:

$$\begin{matrix}{H}_{k} = {f}_{k}^{FEL} ( {X}^{′} ) = [ {f}_{k , RF}^{FEL} ( {X}^{′} ) , {f}_{k , CRF}^{FEL} ( {X}^{′} ) ] \\ = [ ( {c}_{1 , l}^{k , RF} , {c}_{1 , l}^{k , CRF} ) , … , ( {c}_{{n}_{Raw} , l}^{k , RF} , {c}_{{n}_{Raw} , l}^{k , RF} ) , … , ( {c}_{{N}_{Raw} , l}^{k , RF} , {c}_{{N}_{Raw} , l}^{k , CRF} ) ]\end{matrix} \qquad ( 24 )$$

where (c_{1,l}^{k,RF}, c_{1,l}^{k,RF}) represents enhanced mapping on a first sample in new data through the kth hybrid forest group, (cₙ_{_{Raw}}_{,l}^{k,RF},cₙ_{_{Raw}}_{,l}^{k,RF}) represents enhanced mapping on a n_{Raw}th sample in the new data through the kth hybrid forest group, and (c_{N}_{_{Raw}}_{,l}^{k,RF},c_{N}_{_{Raw}}_{,l}^{k,RF}) represents enhanced mapping on a N_{Raw}th sample in the new data through the kth hybrid forest group;
expressing an output H^{K} of the feature incremental layer as follows:

$${H}^{K} = [ {H}_{1} , {H}_{2} , … , {H}_{K} ] ∈ {R}^{{N}_{Raw} \times 2 K} \qquad ( 25 )$$

where H₁ represents a first enhanced feature, H₂ represents a second enhanced feature, and H_{K} represents a K enhanced feature;
expressing a BHFR model without considering the incremental learning strategy as follows:

$$\begin{matrix}Y = {G}^{K} {W}^{K} \\ = [ {Z}_{1} , {Z}_{2} , … , {Z}_{N} \vert {H}_{1} , {H}_{2} , … {H}_{K} ] {W}^{K}\end{matrix} \qquad ( 26 )$$

where G^{K} represents a combination of outputs from the feature mapping layer and the feature incremental layer, that is G^{K}=[Z^{N}|H^{K}], and includes N_{Raw} samples and a (2N+2K) dimensional feature; and W^{K} represents a weight of each of the feature mapping layer and the feature incremental layer relative to the output layer, and is computed as follows:

$${W}^{K} = {{( λ I + {[ {G}^{K} ]}^{T} {G}^{K} )}^{- 1} [ {G}^{K} ]}^{T} Y \qquad ( 27 )$$

where I represents a unit matrix, and represents a regularization term coefficient; and accordingly, expressing a pseudo-inverse computation of G^{K} as:

$$\begin{matrix}{[ {G}^{K} ]}^{†} = {{( λ I + {[ {G}^{K} ]}^{T} G )}^{- 1} [ {G}^{K} ]}^{T} \\ = {[ {Z}^{N} \vert {H}^{K} ]}^{†}\end{matrix} \qquad ( 28 )$$

Further, S4 of constructing the incremental learning layer based on an incremental learning strategy, obtaining a weight matrix with Moore-Penrose pseudo-inverse, and implementing high-precision modeling of the BHFR soft sensor model specifically includes:
obtaining a training subset of the hybrid forest algorithm by performing Bootstrap sampling and RSM sampling on the new data set {X′,y} in a process as follows:

$${\{ {X}_{Bootstrap}^{′ p , j} , {y}_{Bootstrap}^{p , j} \}}_{j = 1}^{J} = {φ}_{p}^{ILL} ( {ϕ}_{p}^{ILL} \{ \{ {X}^{′} , y \} , {P}_{Bootstrap} \} ) \qquad ( 29 )$$

where X′_{Bootstrap}^{p,j} and y_{Bootstrap}^{p,j} represent the input and the output of the J training subset of the hybrid forest algorithm, X′ and y represent the input and the output of the new training set respectively, and ϕᵢ^{ILL}(⋅) and φₚ^{ILL}(⋅) represent Bootstrap sampling and sampling of a pth hybrid forest group in the incremental learning layer;
construction decision trees f_{p,RF}^{ILL}(⋅) and f_{p,CRF}^{ILL}(⋅) in the pth hybrid forest group in the same process (not repeated herein) as the feature mapping layer and the feature incremental layer;
under the condition that one hybrid forest group is added, expressing outputs G^{K+1} of the feature mapping layer, the feature incremental layer and the incremental learning layer as follows:

$$\begin{matrix}{G}^{K + 1} = [ {G}^{K} \vert {f}_{1}^{FEL} ( {X}^{′} ) ] \\ = [ {G}^{K} \vert \{ {f}_{1 , RF}^{FEL} ( {X}^{′} ) , {f}_{1 , CRF}^{FEL} ( {X}^{′} ) \} ] \\ = [ {G}^{K} \vert [ ( {c}_{1 , l}^{1 , RF} , {c}_{1 , l}^{1 , CRF} ) , … , ( {c}_{{N}_{Raw} , l}^{1 , RF} , {c}_{{N}_{Raw} , l}^{1 , CRF} ) ] ]\end{matrix} \qquad ( 30 )$$

where G^{k}=[Zⁿ|H^{k}] includes N_{Raw} samples and a (2N+2K) dimensional feature, and G^{K+1} includes N_{Raw} samples and a (2N+2K+2J) dimensional feature;
recursively updating a Moore-Penrose inverse matrix of G^{K+1} as follows:

$${B}^{T} = \{ \begin{matrix}{[ C ]}^{†} , & if C \neq 0 \\ {[ 1 + {D}^{T} D ]}^{- 1} {{D}^{T} [ {G}^{k} ]}^{†} , & if C = 0\end{matrix} \qquad ( 31 )$$

where a matrix C and a matrix D are computed as follows:

$$C = {H}_{K + 1} - {G}^{K} D \qquad ( 32 )$$

$$D = {[ {G}^{K} ]}^{†} {f}_{1}^{ILL} ( {X}^{New} ) \qquad ( 33 )$$

expressing a recursive formula of the Moore-Penrose inverse matrix of G^{K+1} as follows:

$${[ {G}^{K + 1} ]}^{†} = [ \begin{matrix}{[ {G}^{K} ]}^{†} - {DB}^{T} \\ {B}^{T}\end{matrix} ] \qquad ( 34 )$$

computing an updating matrix W_{K+1} of a weight of the feature mapping layer, the feature incremental layer, the incremental learning layer relative to the output layer as follows:

$${W}^{K + 1} = [ \begin{matrix}{W}^{K} - {DB}^{T} Y \\ {B}^{T} Y\end{matrix} ] \\ where {W}^{K} = {{( λ I + {[ {G}^{K} ]}^{T} {G}^{K} )}^{- 1} [ {G}^{K} ]}^{T} Y ; \qquad ( 35 )$$

implementing rapid incremental learning due to the fact that a pseudo-inverse matrix of the hybrid forest group in the incremental learning layer is merely required to be computed according to a pseudo-inverse updating strategy above;
implementing adaptive incremental learning according to a convergence degree of a training error;
determining a number p of hybrid forest groups of incremental learning by defining a convergence threshold of the error as θ_{Con}, and expressing accordingly an incremental learning training error of the BHFR model as follows:

$$ℓ = {lim}_{p \to ∞} \vert \frac{1}{N} ( \sqrt{{( {G}^{K + p} {W}^{K + p} - y )}^{2}} - \sqrt{{( {G}^{K + p + 1} {W}^{K + p + 1} - y )}^{2}} ) \vert \leq {θ}_{Con} \\ S . T . {θ}_{Con} \geq 0 \qquad ( 36 )$$

where [symbol] represent a training error value between a p+₁ hybrid forest group and a p hybrid forest group of incremental learning, and \sqrt{(G^{K+p}W^{K+p}−y)²} and \sqrt{(G^{K+p+1}W^{K+p+1}−y)²} represent training errors of a BHFR model including p hybrid forest groups and a BHFR model including p+1 hybrid forest groups; and
expressing a predicted output Ŷ of the BHFR soft sensor model as follows:

$$\hat{Y} = {G}^{K + P} {W}^{K + P} \qquad ( 37 )$$

According to a specific embodiment of the present disclosure, the present disclosure has the following technical effects: the BHFR-based soft sensor method for DXN emission in an MSWI process according to the present disclosure constructs a soft sensor model based on the BHFR, and the soft sensor model combines algorithms such as width learning modeling, integrated learning and latent feature extraction: 1) based on a broad learning system (BLS) framework, a non-differential learner is used to construct the soft sensor model including a feature mapping layer, a latent feature extraction layer, a feature incremental layer and an incremental learning layer; 2) internal information of the BHFR model is processed through full information connection, latent feature extraction and mutual information measurement, so as to effectively ensure maximum transmission and minimum redundancy of internal feature information of the BHFR model; 3) a hybrid forest group is used as a mapping unit for incremental learning in a modeling process, and a weight matrix of an output layer is rapidly computed according to a pseudo-inverse strategy, and then the incremental learning is adaptively adjusted based on a convergence degree of a training error, thereby implementing high-precision soft sensor modeling. The validity and rationality of the method are verified by using an industrial process dioxin (DXN) data set.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 is a flowchart of a broad hybrid forest regression (BHFR)-based soft sensor method for DXN emission in a municipal solid waste incineration (MSWI) process according to an embodiment of the present disclosure;

FIG. 2 is a technical flowchart of an MSWI process according to an embodiment of the present disclosure;

FIG. 3 is a training error convergence curve according to an embodiment of the present disclosure;

FIG. 4A is a fitting curve of a training set in a DXN data set according to an embodiment of the present disclosure;

FIG. 4B is a fitting curve of a verification set in a DXN data set according to an embodiment of the present disclosure; and

FIG. 4C is a fitting curve of a testing set in a DXN data set according to an embodiment of the present disclosure.

### DETAILED DESCRIPTION OF THE EMBODIMENTS

The technical solutions of embodiments of the present disclosure will be clearly and completely described below with reference to accompanying drawings. Apparently, the described embodiments are merely some embodiments rather than all embodiments of the present disclosure. All other embodiments derived by a person of ordinary skill in the art based on the embodiments of the present disclosure without creative efforts shall fall within the protection scope of the present disclosure.

An objective of the present disclosure is to provide a broad hybrid forest regression (BHFR)-based soft sensor method for DXN emission in a municipal solid waste incineration (MSWI) process. A soft sensor modeling algorithm based on broad hybrid forest regression (BHFR) is provided with measurement of a dioxin (DXN) emission concentration in the MSWI process as the target.

To make the above objectives, features, and advantages of the present disclosure clearer and more comprehensible, the present disclosure will be further described below in detail with reference to the accompanying drawings and specific implementation modes.

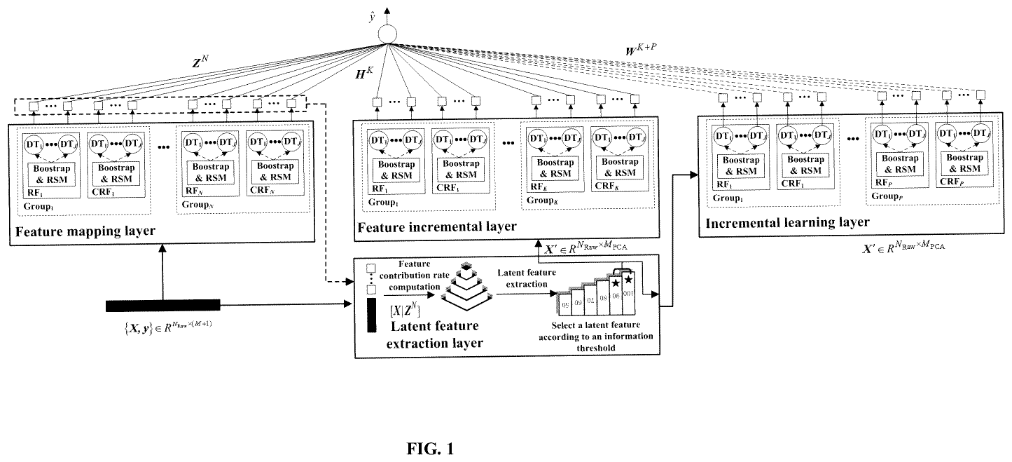

As shown in FIG. 1, the broad hybrid forest regression (BHFR)-based soft sensor method for DXN emission in an MSWI process according to the present disclosure includes:
based on a broad learning system (BLS) framework, a BHFR soft sensor model is constructed for small sample high-dimensional data by replacing a neuron with a non-differential base learner, where the BHFR soft sensor model includes a feature mapping layer, a latent feature extraction layer, a feature incremental layer and an incremental learning layer, and the method specifically includes:
S1, the feature mapping layer is constructed, and a high-dimensional feature is mapped by constructing a hybrid forest group composed of a random forest (RF) and a completely random forest (CRF).

S2, the latent feature extraction layer is constructed, a latent feature is extracted from a feature space of a fully connected hybrid matrix according to a contribution rate, maximum transmission and minimum redundancy of potential valuable information are guaranteed based on an information measurement criterion, and model complexity and computation consumption are reduced.

S3, the feature incremental layer is constructed, and a feature representation capacity is further enhanced by training the feature incremental layer based on an extracted latent feature.

S4, the incremental learning layer is constructed based on an incremental learning strategy, a weight matrix is obtained with a Moore-Penrose pseudo-inverse, and high-precision modeling of the BHFR soft sensor model is implemented.

S5, the soft sensor model is verified by using an industrial process dioxin (DXN) data set.

S6, a soft sensor is used on DXN emission in the MSWI process with the soft sensor model constructed in steps S1-S5.

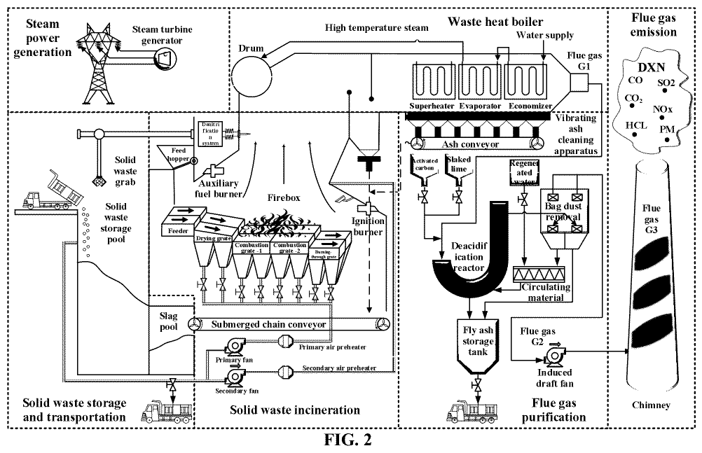

The MSWI process includes technical stages such as solid waste storage and transportation, solid waste incineration, waste heat boilers, steam power generation, flue gas purification and flue gas emission. A grate MSWI process with a daily capacity of 800 tons is taken as an example, and a technical flow is shown in FIG. 2.

An entire process of DXN decomposition, generation, adsorption and emission is combined to describe main functions at each stage as follows:

1) At the solid waste storage and transportation stage: sanitation vehicles transport MSW from various collection stations in a city to an MSWI power plant, and after being weighed and recorded, the MSW is dumped from an unloading platform to an unfermented area in a solid waste storage pool, then is mixed and stirred with a solid waste grab, then is grabbed to a fermentation area, and is fermented and dehydrated for 3-7 days to guarantee a low calorific value of MSW incineration. The research shows that primary MSW contains a small amount of DXN (about 0.8 ng TEQ/Kg) and a variety of chlorine-containing compounds needed for formation of DXN.

2) At the solid waste incineration stage: fermented MSW is thrown by the solid waste grab into a feed hopper, and is pushed into an incinerator through a feeder. After drying, combustion 1, combustion 2 and a burning-through grate in turn, combustible components in the MSW are completely burned, required combustion-supporting air is injected from a lower portion of the grate and a middle portion of a furnace by a primary fan and a secondary fan, and ash produced by final combustion falls from the end of a burning-through grate to a submerged chain conveyor, and is sent to a slag pool after water cooling. In order to ensure that the DXN contained in the original MSW and generated during incineration may be completely decomposed under the high-temperature combustion conditions in the furnace, during furnace combustion, a flue gas temperature is required to be controlled strictly above 850° C., and residence time of high-temperature flue gas in the furnace for more than 2 seconds, so as to guarantee sufficient flue gas turbulence.

3) At the waste heat boiler stage: the high-temperature flue gas (higher than 850° C.) generated in the furnace is pumped into a waste heat boiler system by an induced draft fan, and then passes through a superheater, an evaporator and an economizer. After heat exchange between the high-temperature flue gas and liquid water in a boiler drum, high-temperature steam is generated, such that the high-temperature flue gas is cooled, and a temperature of flue gas (that is, flue gas G1) at an outlet of the waste heat boiler is lower than 200° C. From the perspective of a formation mechanism of DXN, chemical reactions that lead to the formation of DXN when the high-temperature flue gas is cooled by the waste heat boiler include high-temperature gas phase synthesis (800° C.-500° C.), precursor synthesis (450° C.-200° C.) and de novo synthesis (350° C.-250° C.), but there is no unified conclusions yet.

4) At the steam power generation stage: the high-temperature steam generated by the waste heat boiler is used to drive a turbine generator, and mechanical energy is converted into electrical energy, so as to implement self-sufficiency of power consumption at a plant level and on-grid power supply of surplus power, resource recycling and obtain economic benefits.

5) At the flue gas purification stage: the flue gas purification in the MSWI process mainly includes a series of processes, such as denitration (NOX), desulfurization (HCL, HF, SO2, etc.), heavy metal removal (Pb, Hg, Cd, etc.), dioxin adsorption (DXN) and dust removal (particulate matter), so as to satisfy emission standards of incineration flue gas pollutants. It is the most widely used technical means to adsorb DXN in incineration flue gas with an activated carbon injection system, and adsorbed DXN is enriched in fly ash.

6) At the flue gas emission stage: incineration flue gas including trace DXN after cooling and purification (that is, flue gas G2) is sucked by the induced draft fan and emitted into the atmosphere through a chimney. The uninterrupted and long-term running of the MSWI process lead to a large quantity of DXN (that is, memory effects) attached to particles on an inner wall of the chimney. Operation condition for emission is still a research problem at present.

At present, research on the soft sensor of D×N in the MSWI process mainly focuses on measurement of the DXN concentration at an emission stage (that is, flue gas G3), and a research focus of the present application is to construct a soft sensor model at G3 flue gas.

The BHFR modeling strategy according to the present application includes the feature mapping layer, the latent feature extraction layer, the feature incremental layer and the incremental learning layer.

In FIG. 1, {X,y}ϵR^{N}^{_{Raw}}^{×(M+1)} represents original data, where XϵR^{N}^{_{Raw}}^{×M} represents original input data, N_{Raw} represents a number of the original data, M represents a dimension of the original input data, is sourced from six different stages in the MSWI process above, and is collected and stored in a distributed control system (DCS) in seconds, and yϵR^{N}^{_{Raw}}^{×1} represents an output truth of a DXN emission concentration, and is sourced from an emission DXN measurement sample obtained through an off-line measurement method; {DT₁, . . . , DT_{J}} represents J decision tree models in a hybrid forest algorithm, DT₁ represents the first decision tree model and DT_{J} represents the Jth decision tree model; Bootstrap and RSM represent sampling and feature sampling on input data; {RFₙ,CRFₙ} represents the nth hybrid forest group model, and RFₙ and CRFₙ represent the nth RF and CRF models; {Groupₙ}ₙ₌₁^{N} represents that the feature mapping layer includes N hybrid forest group models; Z^{N} represents an output from the feature mapping layer; H^{K} represents an output from the feature incremental layer; [X|Z^{N}] represents a fully connected hybrid matrix consisted by the original data and Z^{N}; X′ϵR^{N}^{_{Raw}}^{×M}^{_{PCA}} represents a new training data after latent feature extraction; {Groupₖ}ₖ₌₁^{P} represents K hybrid forest group models included in the feature incremental layer; {Groupₚ}ₚ₌₁^{P} represents P hybrid forest group model included in the incremental learning layer; and W^{K+P} represents a final weight matrix.

Each part has main functions as follows:

1) The feature mapping layer: the original input data XϵR^{N}^{_{Raw}}^{×M} from six different stages in the MSWI process are subjected to feature mapping through N′ hybrid forest groups in the feature mapping layer {RFₙ,CRFₙ}ₙ₌₁^{N} to obtain a mapping output matrix Z^{N}.

2) The latent feature extraction layer: a latent feature is extracted, through principal component analysis, from the fully connected hybrid matrix [X|Z] composed of the original input data XϵR^{N}^{_{Raw}}^{×M} and the output Z^{N} from the feature mapping layer, redundant information is removed from the feature space, and a latent feature dimension is further determined and a new training set X′ϵR^{N}^{_{Raw}}^{×M}^{_{PM}} is obtained through mutual information between an extracted latent feature and an output truth y of the DXN emission concentration.

3) The feature incremental layer: the new training set X′ϵR^{N}^{_{Raw}}^{×M}_{PM} is taken as an input, feature mapping is performed through K′ hybrid forest groups {RFₖ,CRFₖ}ₖ₌₁^{K} of the feature incremental layer, and an output matrix H^{K} of the enhancement layer is obtained.

4) The incremental learning layer: the new training set X′ϵR^{N}^{_{Raw}}^{×M}^{_{PM}} is taken as an input, and a weight W^{K+P} is gradually increased and updated with the hybrid forest group as the minimum unit until a training error converges.

Essentially, the BHFR replaces a neuron in the original BLS by taking a hybrid forest group composed of the RF and the CRF as the basic mapping unit. The S1, the feature mapping layer is constructed, and a high-dimensional feature is mapped by constructing a hybrid forest group composed of an RF and a CRF includes:
original data are set as {X,y}, where XϵR^{N}^{_{Raw}}^{×M} represents original input data, N_{Raw} represents a number of the original data, M represents a dimension of the original input data, is sourced from six different stages in the MSWI process, and is collected and stored in a distributed control system (DCS) in seconds, and JϵR^{N}^{_{Raw}}^{×1} represents an output truth of a DXN emission concentration, and is sourced from an emission DXN measurement sample obtained through an off-line measurement method; a modeling process of the feature mapping layer is described by taking the nth hybrid forest group of feature mapping layer as an example:
J training subsets of a hybrid forest group model are obtained by performing Bootstrap and random subspace (RSM) sampling on {X,y} as follows:

$${\{ {X}_{Bootstrap}^{n , j} , {y}_{Bootstrap}^{m , j} \}}_{j = 1}^{J} = {φ}_{n}^{FML} ( {ϕ}_{n}^{FML} ( ( X , y ) , {P}_{Bootstrap} ) ) \qquad ( 1 )$$

where X_{Bootstrap}^{n,j} and y_{Bootstrap}^{n,j} represent an input and an output of the jth training subset, ϕₙ^{FML}(⋅) and Pφₙ^{FML}(⋅) represent Bootstrap sampling and RSM sampling of the nth hybrid forest group in the feature mapping layer, and P_{Bootstrap} represents a Bootstrap sampling probability;
a hybrid forest algorithm including J decision trees is trained based on {X_{Bootstrap}^{n,j},y_{Bootstrap}^{n,j}}ⱼ₌₁^{J}, where the jth decision tree of the nth hybrid forest group in the feature mapping layer is expressed as follows:

$${f}_{n , j}^{DT} ( \cdot ) = {\sum }_{l = 1}^{L} {c}_{l} I ( {x}_{Bootstrap}^{n , j} ∈ {R}_{l} ) , l = 1 , 2 , … , L \qquad ( 2 )$$

where L represents a number of leaf nodes of the decision tree, I(⋅) represents an indicator function, and cₗ is computed through recursive splitting;
a splitting loss function Ωᵢ(⋅) of the decision tree in the RF is expressed as:

$${Ω}_{i} ( s , v ) = min ( [ {y}_{L} - E [ {y}_{L} ] ] + [ {y}_{R} - E [ {y}_{R} ] ] ) = min ( {\sum }_{{x}_{Bootstrap}^{n , j} ∈ {R}_{L}} {( {y}_{L}^{i} - {c}_{L} )}^{2} + {\sum }_{{x}_{Bootstrap}^{n , j} ∈ {R}_{R}} {( {y}_{R}^{i} - {c}_{R} )}^{2} ) \qquad ( 3 )$$

where Ωᵢ(s,v) represents a value V of the sth feature value taken as a loss function value of a splitting criterion, y_{L} represents a truth vector of a DXN emission concentration at a left leaf node, E[y_{L}] represents a mathematical expectation of y_{L}, y_{R} represents a truth vector of a DXN emission concentration at a right leaf node, E[y_{R}] represents a mathematical expectation of y_{R}, Y_{L}ⁱ represents the ith DXN emission concentration truth at the left leaf node, y_{R}ⁱ represents the ith DXN emission concentration truth at the right leaf node, c_{L} represents a predicted output of the DXN emission concentration at the left leaf node, and c_{R} represents a predicted output of the DXN emission concentration at the right leaf node;
by minimizing Ωᵢ(s,v), a training set (X_{Bootstrap}^{n,j},y_{Bootstrap}^{m,j}) is split into two tree nodes as follows:

$$min {\{ {Ω}_{i} ( s , v ) \}}_{i = 1}^{{N}_{Raw} \times M} {\to }^{Tree node splitting} \{ \begin{matrix}{R}_{L}^{{N}_{L} \times M} \\ {R}_{R}^{{N}_{R} \times M}\end{matrix} \qquad ( 4 )$$

where R_{L}^{N}^{_{L}}^{×M} and R_{R}^{N}^{_{R}}^{×M} represent sample sets included in a left tree node and a right tree node after division respectively, N_{L} and N_{R} represents a number of samples in R_{L}^{N}^{_{L}}^{×M} and R_{R}^{N}^{_{R}}^{×M} respectively;
predicted output values c_{L}^{RF} and c_{R}^{RF} of the DXN emission concentration at a current left tree node and a current right tree node are expressed as sample truth expectations as follows:

$$\{ \begin{matrix}{c}_{L}^{R F} = E [ {y}_{L} ] , {y}_{L} ∈ {R}_{L}^{{N}_{L} \times M} \\ {c}_{R}^{R F} = E [ {y}_{R} ] , {y}_{R} ∈ {R}_{R}^{{N}_{R} \times M}\end{matrix} \qquad ( 5 )$$

where y_{L} and y_{R} represent truth vectors of the DXN emission concentration in R_{L}^{N}^{_{L}}^{×M} and R_{R}^{N}^{_{R}}^{×M}, and E[y_{L}] and E[y_{R}] represent mathematical expectations of y_{L} and y_{R};
different from the RF, a decision tree in the CRF is split in a completely random selection mode expressed as follows:

$$rand {\{ {( s , v )}_{i} \}}_{i = 1}^{{N}_{Raw} \times M} {\to }^{Tree node splitting} \{ \begin{matrix}{R}_{L}^{{N}_{L} \times M} \\ {R}_{R}^{{N}_{R} \times M}\end{matrix} \qquad ( 6 )$$

where rand{(s,v)ᵢ}ᵢ₌₁^{N}^{_{Raw}}^{×M} represents that a value v of the sth feature is completely randomly selected as a split point;
predicted output values c_{L}^{CRF} and c_{R}^{CRF} of the DXN emission concentration at a left tree node and a right tree node that are randomly split as sample truth expectations are expressed as follows:

$${c}_{L}^{C R F} = E [ {y}_{L} ] , {y}_{L} ∈ {R}_{L}^{{N}_{L} \times M} \qquad ( 7 )$$

$${c}_{R}^{C R F} = E [ {y}_{R} ] , {y}_{R} ∈ {R}_{R}^{{N}_{R} \times M}$$

through the above process, the nth hybrid forest group fₙ^{FML}(⋅) is expressed as follows:

$${f}_{n}^{FML} ( \cdot ) = \{ {f}_{n , RF}^{FML} ( \cdot ) , {f}_{n , CRF}^{FML} ( \cdot ) \} \qquad ( 8 )$$

where f_{n,RF}^{FML}(⋅) represents the nth random forest, and f_{n,CRF}^{FML} represents the nth completely random forest;
the nth mapped feature Zₙ is expressed as

$${Z}_{n} = {f}_{n}^{FML} ( X ) = \{ {f}_{n , RF}^{FML} ( X ) , {f}_{n , CRF}^{FML} ( X ) \} = [ ( {c}_{1 , l}^{n , RF} , {c}_{1 , l}^{n , RF} ) , … , ( {c}_{{n}_{Raw} , l}^{n , RF} , {c}_{{n}_{Raw} , l}^{n , RF} ) , … , ( {c}_{{N}_{Raw} , l}^{n , RF} , {c}_{{N}_{Raw} , l}^{n , RF} ) ] \qquad ( 9 )$$

where (c_{1,l}^{n,RF}, c_{1,l}^{n,RF}) represents a mapped feature, obtained through the nth hybrid forest group, of a first sample of original input data from six different stages in the MSWI process, (cₙ_{_{Raw}}_{,l}^{n,RF},cₙ_{_{Raw}}_{,l}^{n,RF}) represents a mapped feature, obtained through the nth hybrid forest group, of the N_{Raw}th sample of original input data from six different stages in the MSWI process, and (c_{N}_{_{Raw}}_{,l}^{n,RF},c_{N}_{_{Raw}}_{,l}^{n,RF}) represents a mapped feature, obtained through the nth hybrid forest group, of the N_{Raw}th sample of original input data from six different stages in the MSWI process; and
an output of the feature mapping layer is expressed as:

$${Z}^{N} = ( {Z}_{1} , {Z}_{2} , … , {Z}_{N} ) ∈ {R}^{{N}_{R a w} \times 2 N} \qquad ( 10 )$$

where Z₁ represents the first mapped feature, Z₂ represents the second mapped feature, Z_{N} represents the Nth mapped feature, and a mapped feature matrix Z^{N} includes N_{Raw} samples and a 2N dimensional feature.

In order to avoid an over-fitting phenomenon caused by an information loss during information transmission, the BHFR according to the present application uses a full connection strategy to implement information transmission between the feature mapping layer, the feature incremental layer and the incremental learning layer. Moreover, in order to minimize information redundancy during model training, the PCA is used to extract the latent feature from a feature space of the fully connected hybrid matrix, and then mutual information is used to further screen a latent feature related to maximization of truth information, so as to reduce a dimension of high-dimensional data.

S2 that the latent feature extraction layer is constructed, a latent feature is extracted from a feature space of a fully connected hybrid matrix according to a contribution rate, maximum transmission and minimum redundancy of potential valuable information are guaranteed based on an information measurement criterion, and model complexity and computation consumption are reduced specifically includes:
a fully connected hybrid matrix A is obtained and expressed by combining original input data X from six different stages in the MSWI process with a mapped feature matrix Z as follows:

$$A = [ X \vert {Z}^{N} ] ∈ {R}^{{N}_{Raw} \times ( M + 2 N )} \qquad ( 11 )$$

where A includes N_{Raw} samples and a (M+2N) dimensional feature;
that a dimension of A is much higher than a dimension of the original data is considered, redundant information in A is minimized through principal component analysis (PCA), and a correlation matrix R of A is computed as follows:

$$R = \frac{1}{{N}_{R a w} - 1} {A}^{T} A ∈ {R}^{( M + 2 N ) \times ( M + 2 N )} \qquad ( 12 )$$

(M+2N) feature values and corresponding feature vectors are obtained by performing singular value decomposition on R as follows:

$$R = {U}_{( M + 2 N )} {Σ}_{( M + 2 N )} {V}_{( M + 2 N )} \qquad ( 13 )$$

where U_{(M+2N)} represents a (M+2N) order orthogonal matrix, Σ_{(M+2N)} represents a (M+2N) order diagonal matrix, and V_{(M+2N)} represents a (M+2N) order orthogonal matrix;

$${Σ}_{( M + 2 N )} = [ \begin{matrix}{σ}_{1} & & \\ & ⋱ & \\ & & {σ}_{( M + 2 N )}\end{matrix} ] \qquad ( 14 )$$

where σ₁>σ₂> . . . >σ_{(M+2N)} represents eigenvalues arranged in a descending order;
a number of final principal components is determined according to a set latent feature contribution threshold η,

$$η = {\sum }_{q = 1}^{{Q}_{P C A}} {σ}_{q} / {\sum }_{q = 1}^{( M + 2 N )} {σ}_{q} \qquad ( 15 )$$

where a number of latent features is Q_{PCA}«(M+2N);
based on Q_{PCA} determined latent features above, a feature vector matrix V_{Q}_{_{PCA}} (that is, a projection matrix of A) corresponding to a eigenvalue set {σ_{q}}_{q=1}^{Q}^{_{PCA}} is obtained, redundant information is minimized by performing feature projection on A, and an obtained latent feature is recorded as X^{PCA}, that is,

$${X}^{P C A} = A {V}_{{I}_{PCA}} ∈ {R}^{{N}_{R a w} \times {M}_{P C A}} \qquad ( 16 )$$

where V_{Q}_{_{PCA}}ϵR^{(M+2N)×Q}^{_{PCA}} represents a eigenvector of front Q_{PCA} latent features;
a mutual information value I^{MI} between a selected latent feature X^{PCA} and a truth yϵR^{N}^{_{Raw}}^{×1} is computed as follows:

$${I}^{M I} ( {X}^{P C A} , y ) = {\sum }_{q = 1}^{{Q}_{P C A}} p ( {x}_{q}^{P C A} , y ) {log}_{2} \frac{p ( {x}_{q}^{P C A} , y )}{p ( {x}_{q}^{P C A} ) , p ( y )} \qquad ( 17 )$$

where p(x_{q}^{PCA},y) represents a joint probability distribution of the qth latent feature x_{q}^{PCA} and a DXN emission concentration truth y, p(x_{q}^{PCA}) represents a marginal probability distribution of the qth latent feature x_{q}^{PCA}, and p(y) represents a marginal probability distribution of the DXN emission concentration truth y;
a correlation between the selected latent feature and the truth is guaranteed through an information maximization selection mechanism, and the correlation is expressed as:

$${\{ {I}_{q}^{MI} \}}_{q = 1}^{{Q}_{PCA}} {\to }^{{I}_{q}^{M I} \geq ζ} {\{ {I}_{q}^{MI} \}}_{q = 1}^{{Q}_{PCA}^{MI}} \qquad ( 18 )$$

where {I_{q}^{MI}}_{q=1}^{Q} represents a mutual information value between Q_{PCA} latent features x_{q}^{PCA} and the truth y, ζ represents an information maximization threshold, and

$${\{ {I}_{q}^{M I} \}}_{q = 1}^{{Q}_{P C A}^{MI}}$$

represents Q_{PCA}^{MI} latent features having a greatest correlation with information of the DXN emission concentration truth; and
a new data set {X′,y}ϵR^{N}^{_{Raw}}^{×(Q}^{_{PCA}}_{MI}⁺¹⁾ including Q_{PCA}^{MI} latent features is obtained, and a dimension M_{PCA}^{MI}=Q_{PCA}^{MI} after extraction is set.

S3 that the feature incremental layer is constructed, and a feature representation capacity is further enhanced by training the feature incremental layer based on an extracted latent feature specifically includes:
a J training subset of a hybrid forest algorithm is obtained by performing Bootstrap sampling and RSM sampling on the new data set {X′,y} as follows:

$${\{ {X}_{Boostrap}^{′ k , j} , {y}_{Bootstrap}^{k , j} \}}_{j = 1}^{J} = {φ}_{k}^{FEL} ( {ϕ}_{k}^{FEL} ( \{ {X}^{′} , y \} , {P}_{B o o tstrap} ) ) \qquad ( 19 )$$

where X′_{Bootstrap}^{k,j} and y_{Bootstrat}^{k,j} represent an input and an output of the J training subset, X′ and y represent an input and an output of a new training set respectively, ϕₖ^{FEL}(⋅) represents Bootstrap sampling on the kth hybrid forest group, and φₖ^{FEL}(⋅) represents RSM sampling on the kth hybrid forest group;
construction of the jth RF in the kth hybrid forest group is taken as an example as follows:

$$\{ {X}_{Bootstrap}^{′ k , j} , {y}_{B ootstrap}^{k , j} \} {\to }^{{Ω}_{j} ( s , v )} {f}_{k , j}^{D T - P F} ( \cdot ) = {\sum }_{l = 1}^{L} {c}_{l} I ( {x}_{B ootstrap}^{k , j} ∈ {R}_{l} ) , \\ l = 1 , 2 , … , L \qquad ( 20 )$$

where f_{k,j}^{DT-RF}(⋅) represents a jth decision tree of the RF in the kth hybrid forest group in the feature incremental layer; L represents a number of leaf nodes of the decision tree; and cₗ is computed through recursive splitting with specific process formulas of (3)-(5);
an RF model in the kth hybrid forest group in the feature incremental layer is obtained and the RF model is expressed as follows:

$${f}_{k , RF}^{F E L} ( \cdot ) = {\{ {f}_{k , j}^{DT - RF} ( \cdot ) \}}_{j = 1}^{J} \qquad ( 21 )$$

construction of the jth CRF in the kth hybrid forest group is taken as an example similarly as follows:

$$\{ {X}_{Bootstrap}^{′ k , j} , {y}_{B ootstrap}^{k , j} \} {\to }^{ran {d}_{j} ( s , v )} {f}_{k , j}^{DT - CRF} ( \cdot ) = {\sum }_{l = 1}^{L} {c}_{l} I ( {x}_{B ootstrap}^{k , j} ∈ {R}_{l} ) , \\ l = 1 , 2 , … , L \qquad ( 22 )$$

where f_{k,j}^{DT-CRF}(⋅) represents the jth decision tree of the CRF in the kth hybrid forest group in the feature incremental layer; and cₗ is computed through recursive splitting with specific process formulas of (6)-(7);
a CRF model in the kth hybrid forest group in the feature incremental layer is obtained and the CRF model is expressed as follows:

$${f}_{k , CRF}^{F E L} ( \cdot ) = {\{ {f}_{k , j}^{D T - C R F} ( \cdot ) \}}_{j = 1}^{J} \qquad ( 23 )$$

the kth hybrid forest group fₖ^{FEL}(⋅) through the above process is obtained, and the kth enhanced feature is expressed as follows:

$$\begin{matrix}{H}_{k} = {f}_{k}^{FEL} ( {X}^{′} ) = [ {f}_{k , RF}^{F E L} ( {X}^{′} ) , {f}_{k , CRF}^{F E L} ( {X}^{′} ) ] \\ = [ ( {c}_{1 , l}^{k , RF} , {c}_{1 , l}^{k , CRF} ) , … , ( {c}_{{n}_{R a w} , l}^{k , RF} , {c}_{{n}_{R a w} , l}^{k , RF} ) , … , ( {c}_{{N}_{R a w} , l}^{k , RF} , {c}_{{N}_{R a w} , l}^{k , CPF} ) ]\end{matrix} \qquad ( 24 )$$

where (c_{1,l}^{k,RF}, c_{1,l}^{k,RF}) represents enhanced mapping on a first sample in new data through the kth hybrid forest group, (cₙ_{_{Raw}}_{,l}^{k,RF},cₙ_{_{Raw}}_{,l}^{k,RF}) represents enhanced mapping on the n_{Raw}th sample in the new data through the kth hybrid forest group, and (c_{N}_{_{Raw}}_{,l}^{k,RF},c_{N}_{_{Raw}}_{,l}^{k,RF}) represents enhanced mapping on a N_{Raw}th sample in the new data through the kth hybrid forest group;
an output H^{K} of the feature incremental layer is expressed as follows:

$${H}^{K} = [ {H}_{1} , {H}_{2} , … , {H}_{K} ] ∈ {R}^{{N}_{Raw} \times 2 K} \qquad ( 25 )$$

where H₁ represents a first enhanced feature, H₂ represents a second enhanced feature, and H_{K} represents the Kth enhanced feature;
a BHFR model is expressed without considering the incremental learning strategy as follows:

$$Y = {G}^{K} {W}^{K} = [ {Z}_{1} , {Z}_{2} , … , {Z}_{N} \vert {H}_{1} , {H}_{2} , … , {H}_{K} ] {W}^{K} \qquad ( 26 )$$

where G^{K} represents a combination of outputs from the feature mapping layer and the feature incremental layer, that is G^{K}=[Z^{N}|H^{K}], and includes N_{Raw} samples and a (2N+2K) dimensional feature; and W^{K} represents a weight of each of the feature mapping layer and the feature incremental layer relative to the output layer, and is computed as follows:

$${W}^{K} = {{( λ I + {[ {G}^{K} ]}^{T} {G}^{K} )}^{- 1} [ {G}^{K} ]}^{T} Y \qquad ( 27 )$$

where I represents a unit matrix, and λ represents a regularization term coefficient; and a pseudo-inverse computation of G is expressed accordingly as:

$${[ {G}^{K} ]}^{†} = {{( λ I + {[ {G}^{K} ]}^{T} G )}^{- 1} [ {G}^{K} ]}^{T} = {[ {Z}^{N} \vert {H}^{K} ]}^{†} \qquad ( 28 )$$

The BHFR according to the present application takes the hybrid forest group as a basic unit to implement incremental learning according to the convergence degree of a training error. S4 that the incremental learning layer is constructed based on an incremental learning strategy, a weight matrix is obtained with a Moore-Penrose pseudo-inverse, and high-precision modeling of the BHFR soft sensor model is implemented specifically includes:
a training subset of the hybrid forest algorithm is obtained by performing Bootstrap sampling and RSM sampling on the new data set {X′,y} in a process as follows:

$${\{ {X}_{Bootstrap}^{′ p , j} , {y}_{B ootstrap}^{p , j} \}}_{j = 1}^{J} = {φ}_{p}^{ILL} \{ {ϕ}_{p}^{ILL} \{ \{ {X}^{′} , y \} , {P}_{B o o t s t r a p} \} ) \qquad ( 29 )$$

where X′_{Bootstrap}^{p,j} and y_{Bootstrap}^{p,j} represent the input and the output of the jth training subset of the hybrid forest algorithm, X′ and y represent the input and the output of the new training set respectively, and ϕᵢ^{ILL}(⋅) and φₚ^{ILL}(⋅) represent Bootstrap sampling and sampling of a pth hybrid forest group in the incremental learning layer;
decision trees f_{p,RF}^{ILL}(⋅) and f_{p,CRF}^{ILL}(⋅) in the pth hybrid forest group are constructed in the same process (not repeated herein) as the feature mapping layer and the feature incremental layer;
under the condition that one hybrid forest group is added, outputs G^{K+1} of the feature mapping layer, the feature incremental layer and the incremental learning layer are expressed as follows:

$${G}^{K + 1} = [ {G}^{K} \vert {f}_{1}^{F E L} ( {X}^{′} ) ] = [ \text{} {G}^{K} \vert \{ {f}_{1 , FF}^{F E L} ( {X}^{′} ) , {f}_{1 , CRF}^{F E L} ( {X}^{′} ) \} ] = [ \text{} {G}^{K} \vert [ ( {c}_{1 , l}^{1 , RF} , {c}_{1 , l}^{1 , CRF} ) , … , ( {c}_{{N}_{Raw} , l}^{1 , CRF} , {c}_{{N}_{Raw} , l}^{1 , CPF} ) ] ] \qquad ( 30 )$$

where G^{k}=[Zⁿ|H^{k}] includes N_{Raw} samples and a (2N+2K) dimensional feature, and G^{K+1} includes N_{Raw} samples and a (2N+2K+2J) dimensional feature;
a Moore-Penrose inverse matrix of G^{K+1} is updated recursively as follows:

$${B}^{T} = \{ \begin{matrix}{[ C ]}^{†} , & if C \neq 0 \\ {[ 1 + {D}^{T} D ]}^{- 1} {{D}^{T} [ {G}^{k} ]}^{†} , & if C = 0\end{matrix} \qquad ( 31 )$$

where a matrix C and a matrix D are computed as follows:

$$C = {H}_{K + 1} - {G}^{K} D \qquad ( 32 )$$

$$D = {[ {G}^{K} ]}^{†} {f}_{1}^{ILL} ( {X}^{New} ) \qquad ( 33 )$$

a recursive formula of the Moore-Penrose inverse matrix of G^{K+1} is expressed as follows:

$${[ {G}^{K + 1} ]}^{†} = [ \begin{matrix}{[ {G}^{K} ]}^{†} - {DB}^{T} \\ {B}^{T}\end{matrix} ] \qquad ( 34 )$$

an updating matrix W^{K+1} of a weight of the feature mapping layer, the feature incremental layer, the incremental learning layer relative to the output layer is computed as follows:

$${W}^{K + 1} = [ \begin{matrix}{W}^{K} - {DB}^{T} Y \\ {B}^{T} Y\end{matrix} ] where {W}^{K} = {{( λ I + {[ {G}^{K} ]}^{T} {G}^{K} )}^{- 1} [ {G}^{K} ]}^{T} Y ; \qquad ( 35 )$$

rapid incremental learning is implemented due to the fact that a pseudo-inverse matrix of the hybrid forest group in the incremental learning layer is merely required to be computed according to a pseudo-inverse updating strategy above;
adaptive incremental learning is implemented according to a convergence degree of a training error;

a number P of hybrid forest groups of incremental learning is determined by defining a convergence threshold of the error as θ_{Con}, and an incremental learning training error of the BHFR model is expressed accordingly as follows:

$$ℓ = {lim}_{p \to ∞} \vert \frac{1}{N} ( \sqrt{{( {G}^{K + p} {W}^{K + p} - y )}^{2}} - \sqrt{{( {G}^{K + p + 1} {W}^{K + p + 1} - y )}^{2}} ) \vert \leq {θ}_{Con} S . T . {θ}_{C o n} \geq 0 \qquad ( 36 )$$

where [symbol] represent a training error value between a p+1 hybrid forest group and a p hybrid forest group of incremental learning, and \sqrt{(G^{K+p}W^{K+p}−y)²} and \sqrt{(G^{K+p}W^{K+p+1}−y)²} represent training errors of a BHFR model including p hybrid forest groups and a BHFR model including p+1 hybrid forest groups; and

a predicted output Ŷ of the BHFR soft sensor model is expressed as follows:

$$\hat{Y} = {G}^{K + P} {W}^{K + P} \qquad ( 37 )$$

The present application uses actual DXN data of a MSWI power plant for industrial verification. The DXN data are sourced from a MSWI incineration power plant in Beijing, and include 141 sets of DXN emission concentration modeling data from 2009 to 2020. A DXN truth is a converted concentration after 2-hour sampling and testing, and an input variable with missed data and abnormal variables removed is 116 dimensions, and a corresponding value is a mean in a current sampling period of DXN truth.

In the present application, a root mean square error (RMSE), a mean absolute error (MAE) and a coefficient of determination (R2) are selected to compare the performance of different methods with computation as follows:

$$R M S E = \sqrt{{\sum }_{i = 1}^{N} {( {y}_{i} - {\hat{y}}_{i} )}^{2} / ( N - 1 )} \qquad ( 38 )$$

$$MAE = \frac{1}{N} {\sum }_{i = 1}^{N} \vert {\hat{y}}_{i} - {y}_{i} \vert \qquad ( 39 )$$

$${R}^{2} = 1 - {\sum }_{i = 1}^{N} {( {y}_{i} - {\hat{y}}_{i} )}^{2} / {\sum }_{i = 1}^{N} {( {y}_{i} - \overline{y} )}^{2} \qquad ( 40 )$$

where N represents a number of data, yᵢ represents i truth, ŷᵢ represents i predicted value and y̅ represents a mean value.

In the DXN data set, parameters of the BHFR method are set as follows: a minimum sample number Nₛₐₘₚₗₑₛ of leaf nodes of a decision tree is 7, a number of RSM feature selection is \sqrt{N_{features}} a number Nₜᵣₑₑ of decision trees is 10, a number N_{Forest} of hybrid forest groups in each of the feature mapping layer and the feature incremental layer is 10, a latent feature contribution rate threshold η is 0.9, and a regularization parameter λ is 2-10.

A number of latent features for the feature incremental layer and the incremental learning layer is determined based on the fully connected hybrid matrix and a feature space A. A feature dimension of A in a DXN data set is 316. When the latent feature contribution rate threshold η is 0.9, a number of latent features selected from the DXN data set is 35 respectively. Then, mutual information values between 35 latent features and the DXN truth are computed. A mutual information threshold π is set as 0.75, and a number of latent features selected from the DXN data set is 6.

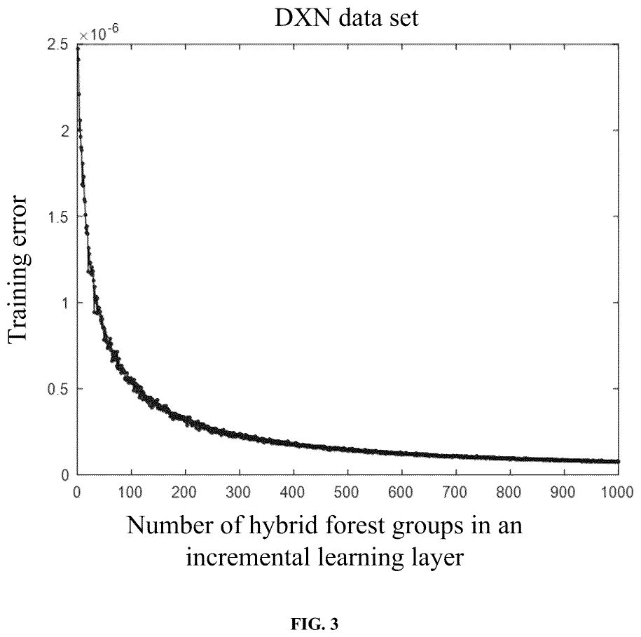

Further, a number of hybrid forest group units in the incremental learning layer is preset to be 1000, and accordingly a relation between the training error of the BHFR model and a number of hybrid forest groups is shown in FIG. 3.

According to a training error curve shown in FIG. 3, a training process of the BHFR on the DXN data set may converge to a certain lower limit.

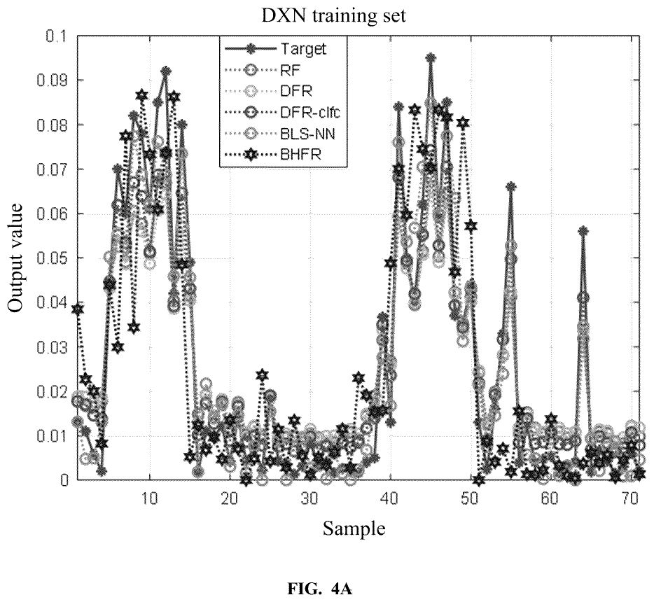

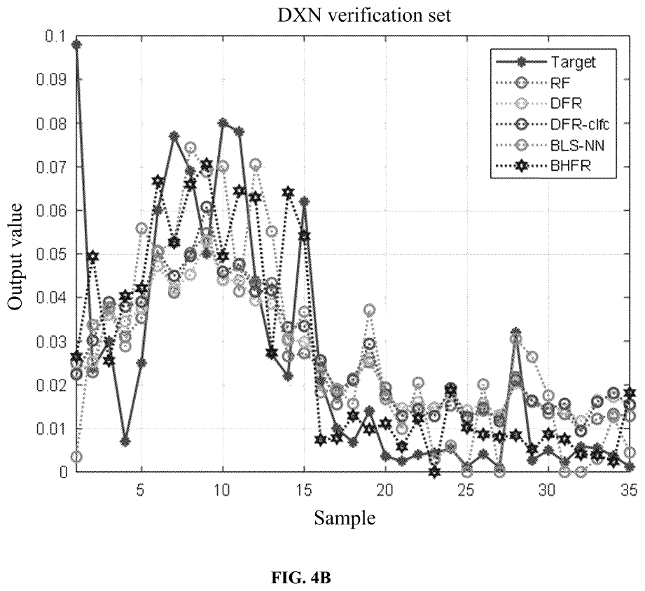

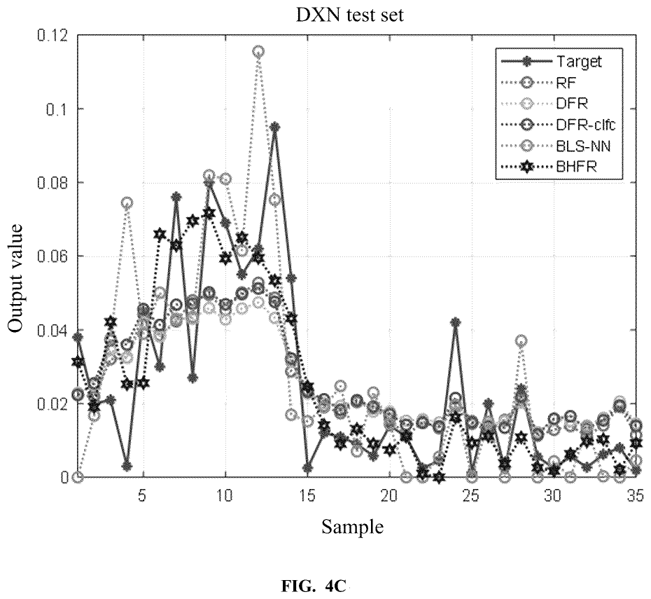

Then, RF, DFR, DFR-clfc and BLS-NN are used to compare with the provided BHFR, and parameters are set as follows: (1) in the case of the RF, a minimum sample number Nₛₘₚₗₑₛ of leaf nodes of a decision tree is 3, a number of RSM feature selection is \sqrt{N_{features}}, and a number Nₜᵣₑₑ of decision trees is 500; (2) in the case of DFR, a minimum sample number Nₛₘₚₗₑₛ of leaf nodes of a decision mode is 3, a number of RSM feature selection is \sqrt{N_{features}}, a number Nₜᵣₑₑ of decision trees is 500, a number NRF of RF models and a number NCRF of CRF models in each layer are 2, and a total number of layers is set as 50; (3) in the case of DFR-clfc, a minimum sample number Nₛₘₚₗₑₛ of leaf nodes of a decision tree is 3, a number of RSM feature selection is \sqrt{N_{features}}, a number Nₜᵣₑₑ of decision trees is 500, a number N_{RF} of RF models and a number N_{CRF} of CRF models in each layer are 2, and a total number of layers is set as 50; and (4) in the case of BLS-NN, a number Nₘ of feature nodes is 5, a number Nₑ of enhanced nodes is 41, a number Nₙ of neurons is 9 and the regularization parameter λ is 2\hat{ }30. The above method is repeated for 20 times under the same conditions, and statistical results and prediction curves are shown in Table 1 and FIGS. 4A-4C.

TABLE 1
Experiment results of the DXN data set

| | | RMSE | | MAE | | R2 | |
|---|---|---|---|---|---|---|---|
| Method | Data set | Mean | Variance | Mean | Variance | Mean | Variance |
| RF | Training set | 1.1159E−02 | 5.7497E−08 | 9.0221E−03 | 4.0684E−08 | 8.5346E−01 | 3.9360E−05 |
| | Verification set | 2.0051E−02 | 1.8026E−07 | 1.4677E−02 | 8.2255E−08 | 5.0196E−01 | 4.3515E−04 |
| | Testing set | 1.6922E−02 | 1.6150E−07 | 1.3548E−02 | 8.9520E−08 | 5.9001E−01 | 3.7817E−04 |
| DFR | Training set | 1.1493E−02 | 8.7413E−09 | 9.4568E−03 | 4.6626E−09 | 8.4463E−01 | 6.3663E−06 |
| | Verification set | 2.0735E−02 | 9.7835E−09 | 1.5780E−02 | 1.1121E−08 | 4.6759E−01 | 2.5813E−05 |
| | Testing set | 1.7791E−02 | 1.7308E−08 | 1.4608E−02 | 1.5235E−08 | 5.4701E−01 | 4.5066E−05 |
| DFR-clfc | Training set | 8.0852E−03 | 2.9078E−06 | 6.6040E−03 | 2.0819E−06 | 9.1986E−01 | 1.1887E−03 |
| | Verification set | 2.0187E−02 | 1.4562E−07 | 1.5626E−02 | 2.3355E−08 | 4.9520E−01 | 3.6404E−04 |
| | Testing set | 1.7025E−02 | 1.5755E−07 | 1.4068E−02 | 6.0233E−08 | 5.8501E−01 | 3.7843E−04 |
| BLS-NN | Training set | 1.2924E−09 | 1.5756E−18 | 9.5358E−10 | 7.2150E−19 | 1.0000E+00 | 8.2358E−29 |
| | Verification set | 6.8845E−02 | 7.0040E−04 | 5.3153E−02 | 3.3474E−04 | −5.6928E+00 | 3.7799E+01 |
| | Testing set | 7.8396E−02 | 6.7692E−04 | 6.0922E−02 | 4.1785E−04 | −8.7153E+00 | 4.7630E+01 |
| BHFR | Training set | 6.0665E−03 | 1.6330E−08 | 3.9665E−03 | 8.4708E−09 | 9.5669E−01 | 3.3481E−06 |
| | Verification set | 2.1551E−02 | 3.5181E−08 | 1.2384E−02 | 3.5083E−08 | 4.2484E−01 | 9.8731E−05 |
| | Testing set | 1.6189E−02 | 2.2474E−08 | 1.1226E−02 | 1.0102E−08 | 6.2491E−01 | 4.8607E−05 |

It may be seen from Table 1 and FIGS. 4A-4C that: 1) RF is superior to DFR in mean statistical results of RMSE, MAE and R2 during training, verification and testing, but inferior to DFR in stability. 2) DFR and DFR-clfc are close to RF in modeling accuracy, and better than RF in modeling stability, DFR-clfc is slightly higher than DFR in accuracy in the training set, the verification set and the testing set, but is inferior to DFR in stability. 3) BLS-NN has obvious over-fitting on the training data, and is worse in generalization performance and stability in the verification set and the testing set, indicating that BLS-NN is difficult to apply to small sample high-dimensional data in a real industrial process in the present application. 4) The BHFR is the best in mean statistical results of RMSE, MAE and R2 indicators in the testing set, and is next to DFR in stability, indicating that BHFR has desirable generalization performance and stability.

To sum up, the DXN soft sensor modeling experiment shows that the BHFR according to the present application is better than classic RF, DFR and improved version DFR-clfc in training and learning capacity, is superior to RF, DFR, DFR-clfc and BLS-NN in modeling accuracy and a data fitting degree in the testing set, and shows obvious advantages in constructing the DXN soft sensor model.

The broad hybrid forest regression (BHFR)-based soft sensor method for DXN emission in an MSWI process according to the present disclosure constructs the soft sensor model based on the BHFR, and the soft sensor model combines algorithms such as broad learning modeling, ensemble learning and latent feature extraction: 1) based on the BLS framework, the non-differential learner is used to construct the soft sensor model including the feature mapping layer, the latent feature extraction layer, the feature incremental layer and the incremental learning layer; 2) internal information of the BHFR model is processed through full information connection, latent feature extraction and mutual information measurement, so as to effectively ensure maximum transmission and minimum redundancy of internal feature information of the BHFR model; 3) the hybrid forest group is used as a mapping unit for incremental learning in a modeling process, and the weight matrix of the output layer is rapidly computed according to the pseudo-inverse strategy, and then the incremental learning is adaptively adjusted based on the convergence degree of the training error, thereby implementing high-precision soft sensor modeling. The validity and rationality of the method are verified by using a high-dimensional an industrial process DXN data set.

Specific examples are used herein to explain the principles and implementation modes of the present disclosure. The foregoing description of the embodiments is merely intended to help understand the method of the present disclosure and its core ideas. Besides, various modifications may be made by a person of ordinary skill in the art to specific embodiments and the scope of application in accordance with the ideas of the present disclosure. In conclusion, the content of the present specification shall not be construed as limitation to the present disclosure.

What is claimed is:

1. A broad hybrid forest regression (BHFR)-based soft sensor method for dioxin (DXN) emission in a municipal solid waste incineration (MSWI) process, comprising: based on a broad learning system (BLS) framework, constructing a BHFR soft sensor model for small sample high-dimensional data by replacing a neuron with a non-differential base learner, wherein the BHFR soft sensor model comprises a feature mapping layer, a latent feature extraction layer, a feature incremental layer and an incremental learning layer, and the method specifically comprises:
S1, constructing the feature mapping layer, and mapping a high-dimensional feature by constructing a hybrid forest group composed of a random forest (RF) and a completely random forest (CRF);
S2, constructing the latent feature extraction layer, extracting a latent feature from a feature space of a fully connected hybrid matrix according to a contribution rate, guaranteeing maximum transmission and minimum redundancy of potential valuable information based on an information measurement criterion, and reducing model complexity and computation consumption;
S3, constructing the feature incremental layer, and further enhancing a feature representation capacity by training the feature incremental layer based on an extracted latent feature;
S4, constructing the incremental learning layer based on an incremental learning strategy, obtaining a weight matrix with a Moore-Penrose pseudo-inverse, and implementing high-precision modeling of the BHFR soft sensor model;
S5, verifying the soft sensor model by using a high-dimensional an industrial process DXN data set; and
S6, using a soft sensor on DXN emission in the MSWI process with the soft sensor model constructed in steps S1-S5.

2. The BHFR-based soft sensor method for DXN emission in an MSWI process according to claim 1, wherein S1 of constructing the feature mapping layer, and mapping a high-dimensional feature by constructing a hybrid forest group composed of a RF and a CRF specifically comprises:
setting original data as {X, y}, wherein XϵR^{N}^{_{Raw}}^{×M} represents original input data, N_{Raw} represents a number of the original data, M represents a dimension of the original input data, is sourced from six different stages in the MSWI process, and is collected and stored in a distributed control system (DCS) in seconds, and yϵR^{N}^{_{Raw}}^{×1} represents an output truth of a DXN emission concentration, and is sourced from an emission DXN measurement sample obtained through an off-line measurement method; describing a modeling process of the feature mapping layer by taking a nth hybrid forest group of feature mapping layer as an example:
obtaining J training subsets of a hybrid forest group model by performing Bootstrap and random subspace (RSM) sampling on {X,y} as follows:
$${\{ {X}_{Bootstrap}^{n , j} , {y}_{Bootstrap}^{n , j} \}}_{j = 1}^{J} = {φ}_{n}^{FML} ( {ϕ}_{n}^{FML} ( ( X , y ) , {P}_{Bootstrap} ) ) \qquad ( 1 )$$
wherein X_{Bootstrap}^{n,j} and y_{Bootstrap}^{n,j} represent an input and an output of a jth training subset respectively, ϕₙ^{FML}(⋅) and φₙ^{FML}(⋅) represent Bootstrap sampling and RSM sampling of the nth hybrid forest group in the feature mapping layer respectively, and P_{Bootstrap} represents a Bootstrap sampling probability;
training a hybrid forest algorithm comprising J decision trees based on Bootstrap {B_{Bootstrap}^{n,j}, y_{Bootstrap}^{n,j}}ⱼ₌₁^{J}, wherein a jth decision tree of the nth hybrid forest group in the feature mapping layer is expressed as follows:
$${f}_{n , j}^{D T} ( \cdot ) = {\sum }_{l = 1}^{L} {c}_{l} I ( {x}_{Bootstrap}^{n , j} ∈ {R}_{l} ) , l = 1 , 2 , … , L \qquad ( 2 )$$
wherein L represents a number of leaf nodes of the decision tree, I(⋅) represents an indicator function, and cₗ is computed through recursive splitting;
expressing a splitting loss function Ωᵢ(⋅) of the decision tree in the RF as:
$${Ω}_{i} ( s , v ) = min ( [ {y}_{L} - E [ {y}_{L} ] ] + [ {y}_{R} - E [ {y}_{R} ] ] ) = min ( {\sum }_{{x}_{Bootstrap}^{n , j} ∈ {R}_{L}} {( {y}_{L}^{i} - {c}_{L} )}^{2} + {\sum }_{{x}_{Bootstrap}^{n , j} ∈ {R}_{R}} {( {y}_{R}^{i} - {c}_{R} )}^{2} ) \qquad ( 3 )$$
wherein Ωᵢ(s,v) represents a value V of a sth feature value taken as a loss function value of a splitting criterion, y_{L} represents a truth vector of a DXN emission concentration at a left leaf node, E[yₗ] represents a mathematical expectation of y_{L}, y_{R} represents a truth vector of a DXN emission concentration at a right leaf node, E[y_{R}] represents a mathematical expectation of y_{R}, y_{L}ⁱ represents a ith DXN emission concentration truth at the left leaf node, y_{R}ⁱ represents a i DXN emission concentration truth at the right leaf node, c_{L} represents a predicted output of the DXN emission concentration at the left leaf node, and c_{R} represents a predicted output of the DXN emission concentration at the right leaf node;
splitting, by minimizing Ωᵢ(s,v), a training set (X_{Bootstrap}^{n,j},y_{Bootstrap}^{n,j}) into two tree nodes as follows:
$$min {\{ {Ω}_{i} ( s , v ) \}}_{i = 1}^{{N}_{Raw} \times M} {\to }^{Tree node splitting} \{ \begin{matrix}{R}_{L}^{{N}_{L} \times M} \\ {R}_{R}^{{N}_{R} \times M}\end{matrix} \qquad ( 4 )$$
wherein R_{L}^{N}^{_{L}}^{×M} and R_{R}^{N}^{_{R}}^{×M} represent sample sets comprised in a left tree node and a right tree node after division respectively, and N_{L} and N_{R} represents a number of samples in R_{L}^{N}^{_{L}}^{×M} and R_{R}^{N}^{_{R}}^{×M} respectively;
expressing predicted output values and c_{L}^{RF} and c_{R}^{RF} of the DXN emission concentration at a current left tree node and a current right tree node as sample truth expectations as follows:
$$\{ \begin{matrix}{c}_{L}^{R F} = E [ {y}_{L} ] , {y}_{L} ∈ {R}_{L}^{{N}_{L} \times M} \\ {c}_{R}^{R F} = E [ {y}_{R} ] , {y}_{R} ∈ {R}_{R}^{{N}_{R} \times M}\end{matrix} \qquad ( 5 )$$
wherein y_{L} and y_{R} represent truth vectors of the DXN emission concentration in R_{L}^{N}^{_{L}}^{×M} and R_{R}^{N}^{_{R}}^{×M}, and E[y_{L}] and E[y_{R}] represent mathematical expectations of y_{L} and y_{R};
different from the RF, expressing a splitting loss function in the CRF in a completely random selection mode as follows:
$$rand {\{ ( s , v ) \}}_{i = 1}^{{N}_{R a w} \times M} {\to }^{Tree node splitting} \{ \begin{matrix}{R}_{L}^{{N}_{L} \times M} \\ {R}_{R}^{{N}_{R} \times M}\end{matrix} \qquad ( 6 )$$
wherein rand{(s,v)ᵢ}ᵢ₌₁^{N}^{_{Raw×M}} represents that a value v of a sth feature is completely randomly selected as a split point;
expressing predicted output values c_{L}^{CRF} and c_{R}^{CRF} of the DXN emission concentration at a left tree node and a right tree node that are randomly split as sample truth expectations as follows:
$${c}_{L}^{C R F} = E [ {y}_{L} ] , {y}_{L} ∈ {R}_{L}^{{N}_{L} \times M} {c}_{R}^{C R F} = E [ {y}_{R} ] , {y}_{R} ∈ {R}_{R}^{{N}_{R} \times M} \qquad ( 7 )$$
expressing, through the above process, the nth hybrid forest group fₙ^{FML}(⋅) as follows:
$${f}_{n}^{FML} ( \cdot ) = \{ {f}_{n , RF}^{FML} ( \cdot ) , {f}_{n , CRF}^{FML} ( \cdot ) \} \qquad ( 8 )$$
wherein f_{n,RF}^{FML}(⋅) represents a nth random forest, and f_{n,CRF}^{FML}(⋅) represents a nth completely random forest;
expressing a nth mapped feature Zₙ as follows:
$$\begin{matrix}{Z}_{n} = {f}_{n}^{FML} ( X ) = \{ {f}_{n , RF}^{FML} ( X ) , {f}_{n , CRF}^{FML} ( X ) \} \\ = [ ( {c}_{1 , l}^{n , RF} , {c}_{1 , l}^{n , RF} ) , … , ( {c}_{{n}_{Raw} , l}^{n , RF} , {c}_{{n}_{Raw} , l}^{n , RF} ) , … , ( {c}_{{N}_{Raw} , l}^{n , RF} , {c}_{{N}_{Raw} , l}^{n , RF} ) ]\end{matrix} \qquad ( 9 )$$
wherein (c_{1,l}^{n,RF}, c_{1,l}^{n,RF}) represents a mapped feature, obtained through the nth hybrid forest group, of a first sample of original input data from six different stages in the MSWI process, (cₙ_{_{Raw}}_{,l}^{n,RF},cₙ_{_{Raw}}_{,l}^{n,RF}) represents a mapped feature, obtained through the nth hybrid forest group, of a n_{Raw}th sample of original input data from six different stages in the MSWI process, and (cₙ_{_{Raw}}_{,l}^{n,RF},cₙ_{_{Raw}}_{,l}^{n,RF}) represents a mapped feature, obtained through the nth hybrid forest group, of a N_{Raw} th sample of original input data from six different stages in the MSWI process; and
expressing an output of the feature mapping layer as:
$${Z}^{N} = ( {Z}_{1} , {Z}_{2} , … , {Z}_{N} ) ∈ {R}^{{N}_{Raw} \times 2 N} \qquad ( 10 )$$
wherein Z₁ represents a first mapped feature, Z₂ represents a second mapped feature, Z_{N} represents a Nth mapped feature, and a mapped feature matrix Z^{N} comprises N_{Raw} samples and a 2N dimensional feature.

3. The BHFR-based soft sensor method for DXN emission in an MSWI process according to claim 2, wherein S2 of constructing the latent feature extraction layer, extracting a latent feature from a feature space of a fully connected hybrid matrix according to a contribution rate, guaranteeing maximum transmission and minimum redundancy of potential valuable information based on an information measurement criterion, and reducing model complexity and computation consumption specifically comprises:
obtaining and expressing a fully connected hybrid matrix A by combining original input data X from six different stages in the MSWI process with a mapped feature matrix Z^{N} as follows:
$$A = [ X \vert {Z}^{N} ] ∈ {R}^{{N}_{Raw} \times ( M + 2 N )} \qquad ( 11 )$$
wherein A comprises N_{Raw} samples and a (M+2N) dimensional feature;
considering that a dimension of A is much higher than a dimension of the original data, minimizing redundant information in A through principal component analysis (PCA), and computing a correlation matrix R of A as follows:
$$R = \frac{1}{{N}_{Raw} - 1} {A}^{T} A ∈ {R}^{( M + 2 N ) \times ( M + 2 N )} \qquad ( 12 )$$
obtaining (M+2N) feature values and corresponding feature vectors by performing singular value decomposition on R as follows:
$$R = {U}_{( M + 2 N )} {\sum }_{( M + 2 N )} {V}_{( M + 2 N )} \qquad ( 13 )$$
wherein U_{(M+2N)} represents a (M+2N) order orthogonal matrix, Σ_{(M+2N)} represents a (M+2N) order diagonal matrix, and V_{(M+2)} represents a (M+2N) order orthogonal matrix;
$${\sum }_{( M + 2 N )} = [ \begin{matrix}{σ}_{1} & & \\ & ⋱ & \\ & & {σ}_{( M + 2 N )}\end{matrix} ] \qquad ( 14 )$$
wherein σ₁>σ₂> . . . >σ_{(M+2N)} represents eigenvalues arranged in a descending order;
determining a number of final principal components according to a set latent feature contribution threshold η,
$$η = {\sum }_{q = 1}^{{Q}_{PCA}} {σ}_{q} / {\sum }_{q = 1}^{( M + 2 N )} {σ}_{q} \qquad ( 15 )$$
wherein a number of latent features is Q_{PCA}<<(M+2N);
based on Q_{PCA} determined latent features above, obtaining a feature vector matrix V_{Q}_{_{PCA}} (that is, a projection matrix of A) corresponding to a eigenvalue set {σ_{q}=}_{q=1}^{Q}^{_{PCA}}, minimizing redundant information by performing feature projection on A, and recording an obtained latent feature as X^{PCA}, that is,
$${X}^{PCA} = {AV}_{{I}_{PCA}} ∈ {R}^{{N}_{Raw} \times {M}_{PCA}} \qquad ( 16 )$$
wherein V_{Q}_{_{PCA}}ϵR^{(M+2N)×Q}^{_{PCA}} represents a eigenvector of front Q_{PCA} latent features;
computing a mutual information value I^{MI} between a selected latent feature X^{PCA} and a truth yϵR^{N}^{_{Raw}}^{×1} as follows:
$${I}^{MI} ( {X}^{PCA} , y ) = {\sum }_{q = 1}^{{Q}_{PCA}} p ( {x}_{q}^{PCA} , y ) {log}_{2} \frac{p ( {x}_{q}^{PCA} , y )}{p ( {x}_{q}^{PCA} ) , p ( y )} \qquad ( 17 )$$
wherein p(x_{q}^{PCA},y) represents a joint probability distribution of a qth latent feature x_{q}^{PCA} and a DXN emission concentration truth y, p(x_{q}^{PCA}) represents a marginal probability distribution of the 9th latent feature x_{q}^{PCA}, and p(y) represents a marginal probability distribution of the DXN emission concentration truth y;
guaranteeing a correlation between the selected latent feature and the truth through an information maximization selection mechanism, and expressing the correlation as:
$${\{ {I}_{q}^{MI} \}}_{q = 1}^{{Q}_{PCA}} {⟶}^{{I}_{q}^{MI} \geq ζ} {\{ {I}_{q}^{MI} \}}_{q = 1}^{{Q}_{PCA}^{MI}} ; \qquad ( 18 )$$
wherein {I_{q}^{MI}}_{q=1}^{Q} represents a mutual information value between Q_{PCA} latent features x_{q}^{PCA} and the truth y, ζ represents an information maximization threshold, and
$${\{ {I}_{q}^{MI} \}}_{q = 1}^{{Q}_{PCA}^{MI}}$$
represents latent features having a greatest correlation with information of the DXN emission concentration truth y; and
obtaining a new data set {X′,y}ϵR^{N}^{_{Raw}}^{×(Q}^{_{PCA}}^{^{MI}}⁺¹⁾ comprising Q_{PCA}^{MI} latent features, and setting a dimension M_{PCA}^{NI}=Q_{PCA}^{MI} after extraction.

4. The BHFR-based soft sensor method for DXN emission in an MSWI process according to claim 3, wherein S3 of constructing the feature incremental layer, and further enhancing a feature representation capacity by training the feature incremental layer based on an extracted latent feature specifically comprises:
obtaining a J training subset of a hybrid forest algorithm by performing Bootstrap sampling and RSM sampling on the new data set {X′,y} as follows:
$${\{ {X}_{Bootstrap}^{′ k , j} , {y}_{Bootstrap}^{k , j} \}}_{j = 1}^{J} = {φ}_{k}^{FEL} ( {ϕ}_{k}^{FEL} ( \{ {X}^{′} , y \} , {P}_{Bootstrap} ) ) \qquad ( 19 )$$
wherein X′_{Bootstrap}^{k,j} and y_{Bootstrap}^{k,j} represent an input and an output of the jth training subset, X′ and y represent an input and an output of a new training set respectively, ϕₖ^{FEL}(⋅) represents Bootstrap sampling on a kth hybrid forest group, and φₖ^{FEL}(⋅) represents RSM sampling on the kth hybrid forest group;
taking construction of a jth RF in the kth hybrid forest group as an example as follows:
$$\{ {X}_{Bootstrap}^{′ k , j} , {y}_{Bootstrap}^{k , j} \} {⟶}^{{Ω}_{j} ( s , v )} {f}_{k , j}^{DT - RF} ( \cdot ) = {\sum }_{l = 1}^{L} {c}_{l} I ( {x}_{Bootstrap}^{k , j} ∈ {R}_{l} ) , \text{} l = 1 , \text{} 2 , ... , L \qquad ( 20 )$$
wherein f_{k,j}^{DT-RF}(⋅) represents a jth decision tree of the RF in the kth hybrid forest group fDT-RF (⋅) in the feature incremental layer; L represents a number of leaf nodes of the decision tree; and cₗ is computed through recursive splitting with specific process formulas of (3)-(5);
obtaining an RF model in the kth hybrid forest group in the feature incremental layer and expressing the RF model as follows:
$${f}_{k , RF}^{FEL} ( \cdot ) = {\{ {f}_{k , j}^{DT - REF} ( \cdot ) \}}_{j = 1}^{J} \qquad ( 21 )$$
similarly taking construction of a jth CRF in the kth hybrid forest group as an example as follows:
$$\{ {X}_{Bootstrap}^{′ k , j} , {y}_{Bootstrap}^{k , j} \} {⟶}^{{rand}_{j} ( s , v )} {f}_{k , j}^{DT - CRF} ( \cdot ) = {\sum }_{l = 1}^{L} {c}_{l} I ( {x}_{Bootstrap}^{k , j} ∈ {R}_{l} ) , \\ l = 1 , 2 , … , L \qquad ( 22 )$$
wherein f_{k,j}^{DT-CRF}(⋅) represents a jth decision tree of the CRF in the kth hybrid forest group in the feature incremental layer; and cₗ is computed through recursive splitting with specific process formulas of (6)-(7);
obtaining a CRF model in the kth hybrid forest group in the feature incremental layer and expressing the CRF model as follows:
$${f}_{k , CRF}^{FEL} ( \cdot ) = {\{ {f}_{k , j}^{DT - CRF} ( \cdot ) \}}_{j = 1}^{J} \qquad ( 23 )$$
obtaining the kth hybrid forest group fₖ^{FEL}(⋅) through the above process, and expressing a kth enhanced feature as follows:
$$\begin{matrix}{H}_{k} = {f}_{k}^{FEL} ( {X}^{′} ) = [ {f}_{k , RF}^{FEL} ( {X}^{′} ) , {f}_{k , CRF}^{FEL} ( {X}^{′} ) ] \\ = [ ( {c}_{1 , l}^{k , RF} , {c}_{1 , l}^{k , CRF} ) , … , ( {c}_{{n}_{Raw} , l}^{k , RF} , {c}_{{n}_{Raw} , l}^{k , RF} ) , … , ( {c}_{{N}_{Raw} , l}^{k , RF} , {c}_{{N}_{Raw} , l}^{k , CRF} ) ]\end{matrix} \qquad ( 24 )$$
wherein (c_{1,l}^{n,RF}, c_{1,l}^{n,RF}) represents enhanced mapping on a first sample in new data through the kth hybrid forest group, (cₙ_{_{Raw}}_{,l}^{k,RF},cₙ_{_{Raw}}_{,l}^{k,RF}) represents enhanced mapping on a n_{Raw}th sample in the new data through the kth hybrid forest group, and (c_{N}_{_{Raw}}_{,l}^{k,RF},c_{N}_{_{Raw}}_{,l}^{k,RF}) represents enhanced mapping on a N_{Raw}th sample in the new data through the kth hybrid forest group;
expressing an output Hₖ of the feature incremental layer as follows:
$${H}^{K} = [ {H}_{1} , {H}_{2} , … , {H}_{K} ] ∈ {R}^{{N}_{Raw} \times 2 K} \qquad ( 25 )$$
wherein H₁ represents a first enhanced feature, H₂ represents a second enhanced feature, and H_{K} represents a Kth enhanced feature;
expressing a BHFR model without considering the incremental learning strategy as follows:
$$\begin{matrix}Y = {G}^{K} {W}^{K} \\ = [ {Z}_{1} , {Z}_{2} , … , {Z}_{N} \vert {H}_{1} , {H}_{2} , … {H}_{K} ] {W}^{K}\end{matrix} \qquad ( 26 )$$
wherein G^{K} represents a combination of outputs from the feature mapping layer and the feature incremental layer, that is G^{K}=[Z^{N}|H^{K}], and comprises N_{Raw} samples and a (2N+2K) dimensional feature; and W^{K} represents a weight of each of the feature mapping layer and the feature incremental layer relative to the output layer, and is computed as follows:
$${W}^{K} = {{( λ I + {[ {G}^{K} ]}^{T} {G}^{K} )}^{- 1} [ {G}^{K} ]}^{T} Y \qquad ( 27 )$$
wherein I represents a unit matrix, and λ represents a regularization term coefficient; and accordingly, expressing a pseudo-inverse computation of G^{K} as:
$$\begin{matrix}{[ {G}^{K} ]}^{†} = {{( λ I + {[ {G}^{K} ]}^{T} G )}^{- 1} [ {G}^{K} ]}^{T} \\ = {[ {Z}^{N} \vert {H}^{K} ]}^{†}\end{matrix} . \qquad ( 28 )$$

5. The BHFR-based soft sensor method for DXN emission in an MSWI process according to claim 4, wherein S4 of constructing the incremental learning layer based on an incremental learning strategy, obtaining a weight matrix with Moore-Penrose pseudo-inverse, and implementing high-precision modeling of the BHFR soft sensor model specifically comprises:
obtaining a training subset of the hybrid forest algorithm by performing Bootstrap sampling and RSM sampling on the new data set {X′,y} in a process as follows:
$${\{ {X}_{Bootstrap}^{′ p , j} , {y}_{Bootstrap}^{p , j} \}}_{j = 1}^{J} = {φ}_{p}^{ILL} ( {ϕ}_{p}^{ILL} \{ \{ {X}^{′} , y \} , {P}_{Bootstrap} \} ) \qquad ( 29 )$$
wherein Bootstrap X′_{Bootstrap}^{p,j} and y_{Bootstrap}^{p,j} represent the input and the output of the jth training subset of the hybrid forest algorithm, X′ and y represent the input and the output of the new training set respectively, and ϕᵢ^{ILL}(⋅) and φₚ^{ILL}(⋅) represent Bootstrap sampling and sampling of a pth hybrid forest group in the incremental learning layer;
construction decision trees f_{p,RF}^{ILL}(⋅) and f_{p,CRF}^{ILL}(⋅) in the pth hybrid forest group in the same process (not repeated herein) as the feature mapping layer and the feature incremental layer;
under the condition that one hybrid forest group is added, expressing outputs G^{K+1} of the feature mapping layer, the feature incremental layer and the incremental learning layer as follows:
$$\begin{matrix}{G}^{K + 1} = [ {G}^{K} \vert {f}_{1}^{FEL} ( {X}^{′} ) ] \\ = [ {G}^{K} \vert \{ {f}_{1 , RF}^{FEL} ( {X}^{′} ) , {f}_{1 , CRF}^{FEL} ( {X}^{′} ) \} ] \\ = [ {G}^{K} \vert [ ( {c}_{1 , l}^{1 , RF} , {c}_{1 , l}^{1 , CRF} ) , … , ( {c}_{{N}_{Raw} , l}^{1 , RF} , {c}_{{N}_{Raw} , l}^{1 , CRF} ) ] ]\end{matrix} \qquad ( 30 )$$
wherein G^{k}=[Zⁿ|H^{k}] comprises N_{Raw} samples and a (2N+2K) dimensional feature, and G^{K+1} comprises N_{Raw} samples and a (2N+2K+2J) dimensional feature;
recursively updating a Moore-Penrose inverse matrix of G^{K+1} as follows:
$${B}^{T} = \{ \begin{matrix}{[ C ]}^{†} , & if C \neq 0 \\ {[ 1 + {D}^{T} D ]}^{- 1} {{D}^{T} [ {G}^{k} ]}^{†} , & if C = 0\end{matrix} \qquad ( 31 )$$
wherein a matrix C and a matrix D are computed as follows:
$$C = {H}_{K + 1} - {G}^{K} D \qquad ( 32 )$$
$$D = {[ {G}^{K} ]}^{†} {f}_{1}^{ILL} ( {X}^{New} ) \qquad ( 33 )$$
expressing a recursive formula of the Moore-Penrose inverse matrix of G^{K+1} as follows:
$${[ {G}^{K + 1} ]}^{†} = [ \begin{matrix}{[ {G}^{K} ]}^{†} - {DB}^{T} \\ {B}^{T}\end{matrix} ] \qquad ( 34 )$$
computing an updating matrix W^{K+1} of a weight of the feature mapping layer, the feature incremental layer, the incremental learning layer relative to the output layer as follows:
$${W}^{K + 1} = [ \begin{matrix}{W}^{K} - {DB}^{T} Y \\ {B}^{T} Y\end{matrix} ] \\ wherein {W}^{K} = {{( λ I + {[ {G}^{K} ]}^{T} {G}^{K} )}^{- 1} [ {G}^{K} ]}^{T} Y ; \qquad ( 35 )$$
implementing rapid incremental learning due to the fact that a pseudo-inverse matrix of the hybrid forest group in the incremental learning layer is merely required to be computed according to a pseudo-inverse updating strategy above;
implementing adaptive incremental learning according to a convergence degree of a training error;
determining a number P of hybrid forest groups of incremental learning by defining a convergence threshold of the error as θ_{Con}, and expressing accordingly an incremental learning training error of the BHFR model as follows:
$$ℓ = {lim}_{p \to ∞} \vert \frac{1}{N} ( \sqrt{{( {G}^{K + p} {W}^{K + p} - y )}^{2}} - \sqrt{{( {G}^{K + p + 1} {W}^{K + p + 1} - y )}^{2}} ) \vert \leq {θ}_{Con} \\ S . T . {θ}_{Con} \geq 0 \qquad ( 36 )$$
wherein [symbol] represent a training error value between a p+1 hybrid forest group and a p hybrid forest group of incremental learning, and \sqrt{(G^{K+p}W^{K+p}−y)²} and \sqrt{(G^{K+p+1}W^{K+p+1}−y)²} represent training errors of a BHFR model comprising P hybrid forest groups and a BHFR model comprising p+1 hybrid forest groups; and
expressing a predicted output Ŷ of the BHFR soft sensor model as follows:
$$\hat{Y} = {G}^{K + P} {W}^{K + P} . \qquad ( 37 )$$
